(19) United States
(12) Patent Application Publication
(10) Pub. No.: US 2017/0106132 A1
(43) Pub. Date: Apr. 20, 2017

(54) BONE BYPASS SHUNTS AND METHODS USING THEREOF

(71) Applicant: XEREM MEDICAL LTD, Tel-Aviv (IL)

(72) Inventors: Morris LASTER, Jerusalem (IL); Sigal KREMER-TAL, Ramat Hasharon (IL)

(21) Appl. No.: 15/394,141

(22) Filed: Dec. 29, 2016

Related U.S. Application Data
(63) Continuation-in-part of application No. 14/368,537, filed on Jun. 25, 2014.
(60) Provisional application No. 62/272,171, filed on Dec. 29, 2015.

(51) Int. Cl. A61M 1/34; A61M 1/36; A61M 1/38; A61M 1/16

(52) U.S. Cl. CPC A61M 1/34; A61M 1/1678; A61M 1/3486; A61M 1/3489; A61M 1/3639; A61M 1/38; A61M 2205/7545; A61M 2210/02; A61M 1/3655

(57) ABSTRACT

Bone bypass shunts and methods using thereof. Method for affecting a pathophysiological condition in a body of a live subject includes exemplary steps/procedures of: connecting an inlet port of a bone bypass shunt to a first bone portion adjacent a first bone marrow location such that inlet port lumen of inlet port facilitates fluid communication with blood accumulated or flowing at first blood marrow location; connecting outlet port of the bone bypass shunt to second bone portion adjacent second bone marrow location, such that outlet port lumen of outlet port facilitates fluid communication with bone marrow; a formed cavity, or/and bone marrow vasculature, located at second bone marrow location; via inlet port lumen, removing a chosen volume of blood from first bone marrow location; and via outlet port lumen, delivering chosen volume of blood to the second bone marrow location.

### CROSS-REFERENCE TO RELATED APPLICATIONS

This is a Non-Provisional Application of U.S. Provisional Patent Application Ser. No. 62/272,171, titled “Bypass-Shunts to Bone Marrow Blood,” filed on Dec. 29, 2015; and is a Continuation-in-Part of U.S. National Phase patent application Ser. No. 14/368,537, titled “System and Method for Blood Filtering or/and Treatment,” filed on Jun. 25, 2014, whose priorities are claimed, and which are hereby incorporated by reference as if fully set forth herein in their entireties.

### FIELD AND BACKGROUND OF THE INVENTION

The present invention relates to apparatuses and methods for recirculating bone marrow blood, and more specifically, to use of bone bypass shunts for affecting (changing, treating or/and maintaining) a pathophysiological condition in a body of a live subject. The pathophysiological condition may be a systemic or/and a local type of pathophysiological condition, and affecting the pathophysiological condition may involve filtering or/and treating blood, by delivering medication, or/and by controlling dynamic properties of the recirculated bone marrow blood.

Bone marrow is a spongy tissue residing within bones. Bone marrow is the major source of both hematopoietic and mesenchymal stem/progenitor cells and their derivatives recruited and homing to injured tissue for the purpose of ensuring hematopoietic and skeletal homeostasis. It has been established that bone marrow contributes to systemic tissue homeostasis. As such, intrinsic aberrations in bone marrow homeostasis may represent a major cause for many pathophysiological conditions, such as osteoporosis, osteodystrophy, osteopetrosis, bone marrow dysplasia, abnormal wound healing, chronic inflammation, systemic diseases, and pathophysiological syndromes, such as systemic fibroses, infections, obstructions, and multiple organ failures. However, the relationship between bone marrow homeostasis along with its physiological environment, and pathophysiological conditions, remains controversial and paradoxical.

Many bone marrow-related, local as well as systemic, diseases or conditions remain incurable. There is thus an on-going need for developing and practicing new or/and improved, and efficient, therapeutic methodologies for treating bone marrow associated pathophysiological conditions.

Congestive heart failure (CHF) is a condition in which the blood pumping function of the heart is inadequate to meet the needs of body tissue. CHF is one of the most common causes of hospitalization and mortality in Western society.

CHF results from a weakening or stiffening of the heart muscle most commonly caused by myocardial ischemia (due to, for example, myocardial infarction) or cardiomyopathy (e.g. myocarditis, amyloidosis). Such weakening or stiffening leads to reduced cardiac output, an increase in cardiac filling pressures, and fluid accumulation. Reduced cardiac output can lead to diminished kidney function and a result to retention of fluid in body tissue. The lungs and liver may also become congested with fluid, thereby impairing the normal function of these organs. In addition, the intestines may become less efficient in absorbing nutrients and medicines. CHF may result in refractory pulmonary edema which may be treated by IV diuresis or ultrafiltration via hemofiltration or dialysis. Over time, untreated CHF will negatively affect virtually every organ in the body.

End-Stage Renal Disease (ESRD) occurs when the kidneys are no longer able to function at a level that is necessary for day-to-day life, up to the point where kidney function is less than about 10% of a normal, disease-free kidney. The most common cause of ESRD is diabetes. Symptoms of ESRD can include, for example, unintentional weight loss, nausea or vomiting, general ill feeling, fatigue, headache, decreased urine output, easy bruising or bleeding, blood in the vomit or stools, elevated blood urea nitrogen (BUN) levels and decreased creatinine clearance.

Dialysis and/or ultrafiltration are performed on individuals suffering from ESRD or CHF -related fluid overload. The process involves removing waste substances and fluid from the blood that are normally eliminated by the kidneys. Dialysis may also be used in individuals exposed to toxic substances in order to prevent renal failure from occurring.

There are two types of dialysis that may be performed: hemodialysis and peritoneal dialysis.

Hemodialysis involves fluid removal through ultrafiltration, causing free water and some dissolved solutes to move across the membrane along a created pressure gradient. Hemodialysis also utilizes a counter current flow of dialysate which performs adjustments of solutes by creating concentration gradients across the membrane at a maximum and increases the efficiency of the dialysis. The blood is taken by a special type of access, called an arteriovenous (AV) fistula, which is placed surgically, usually in the arm. After access has been established, the blood drains though a large hemodialysis machine which bathes the hemofiltration cartridge in a special dialysate solution that adjusts solute concentration and removes waste substances and fluid. The “clean” blood is then returned to the bloodstream. Hemodialysis is usually performed three times a week with each treatment lasting from 3 to 5 or more hours. Because proper maintenance of hemodialysis equipment (e.g. membranes, pumps) is critical, hemodialysis sessions are often performed at a treatment center. Possible complications of hemodialysis can include muscle cramps and low blood pressure caused by removing too much fluid and/or removing fluid too rapidly. The AY fistula often undergoes thrombosis which limits the use of the fistula and may require surgical interventions for clearing and or replacement of the fistula.

Peritoneal dialysis uses the peritoneal membrane to filter the blood. Peritoneal dialysis is performed by surgically placing a special, soft, hollow lube into the lower abdomen near the navel. A mixture of minerals and sugar dissolved in water, called dialysate solution, is instilled into the peritoneal cavity and is left in the abdomen for a designated period of time in which the dialysate fluid absorbs the waste products, toxins and extra water through the peritoneum membranes. After several hours, the used solution containing the wastes from the blood is drained from the abdomen through the tube. Then the abdomen is refilled with fresh dialysis solution, arid the cycle is repeated. The process of draining and refilling is called an exchange. Patients usually undergo four to six exchanges of the dialysis solution per day. An infection of the peritoneum, or peritonitis, is the most common problem of peritoneal dialysis.

Although dialysis is a common procedure it suffers from several disadvantages, including fluids balance impairment, the need of special diet, high blood pressure, psychological problems because of the change in the life style due the need to go to the dialysis treatment several times a week for several hours each time. Due to the increasing numbers of patients requiring dialysis, this introduces a tremendous burden on the healthcare system.

Several attempts have been made to devise systems which overcome at least some of the aforementioned limitations of dialysis devices. U.S. Pat. No. 5,037,385 and Ser. No. 10/922,478 disclose implantable peritoneal dialysis devices. The aforementioned system described includes an implantable peritoneourinary pump system and an implantable dialysate infusion system. When in use, the de vice has a semi-permeable reservoir implanted in the peritoneal cavity. The reservoir receives blood waste and drains through one or more conduits via a pump to the biological bladder, which is a complicated arrangement.

U.S. Pat. No. 5,902,336 describes another implantable system which employs an ultrafiltration device for removing low to medium molecular weight solutes and fluids from the blood of a patient experiencing renal failure. In this system the fluid flows between the patient's vascular system, through an access to the artery and/or vein, and the patient's bladder or urethra. As such, this system requires surgical attachment of a metal or hard plastic device, to a soft biological tissue (artery or vein), a procedure which often results in undesirable side effects such as vessel shearing/tearing, clotting, fibrosis, infection and thrombosis.

U.S. Patent Appl. Pub. No. US 2014/0358060 A1, by the same applicant/assignee of the present invention, discloses techniques (systems, methods) for filtering or treating blood of a subject. The system includes a bone port for establishing fluid communication with a bone marrow of the bone and a return port for returning blood from the bone marrow to a circulation of the subject. The system further includes a blood treatment or filtering device interposed between the bone and return, ports thereby establishing a mini-circulatory system.

The present disclosure extends the scope of, and provides additional exemplary embodiments to, the US 2014/0358060 disclosure.

### SUMMARY OF THE INVENTION

According to one aspect of the present invention there is provided a system for filtering blood of a subject comprising: (a) a bone port configured for establishing fluid communication with a bone marrow of the bone; (b) a fluid fractioning device being in fluid communication with the bone port and being capable of selectively fractioning blood flowing out of the bone marrow to thereby retain a fraction of the blood; and (c) a return port being in fluid communication with the fluid fractioning device and being for returning a non-retained fraction of the blood to a circulation of the subject.

According to further features in exemplary embodiments of the invention described below, the system further comprises a fluid conduit for routing the fraction of the blood retained by the fluid fractioning device to a bladder, a Genito-Urinary system or a reservoir.

According to still further features in the described exemplary embodiments the system further comprises a device for increasing a blood pressure gradient across the bone port.

According to still further features in the described exemplary embodiments the system further comprises a device for increasing a blood pressure gradient across the fluid fractioning device.

According to still further features in the described exemplary embodiments the bone port includes at least one elongated cylinder having a central bore.

According to still further features in the described exemplary embodiments the fluid fractioning device includes at least one filter.

According to still further features in the described exemplary embodiments the filter is impermeable to molecules above a predetermined size and permeable to water and solutes of the blood.

According to still further features in the described exemplary embodiments the molecules are 10-500 kDa.

According to still further features in the described exemplary embodiments the device is a pump.

According to still further features in the described exemplary embodiments the pump is a peristaltic pump.

According to still further features in the described exemplary embodiments the system further comprises a mechanism for minimizing clogging of the filter.

According to still further features in the described exemplary embodiments the mechanism is configured for creating an electrical field at or near the filter.

According to still further features in the described exemplary embodiments the electrical field is an alternating current (AC) field.

According to still further features in the described exemplary embodiments the mechanism for minimizing clogging is a blowback mechanism.

According to still further features in the described exemplary embodiments (c) is effected by returning the non-retained fraction of the blood to the bone marrow of the bone.

According to another aspect of the present invention there is provided a method of filtering blood of a subject comprising: (a) communicating blood from a bone marrow of a bone of the subject to a fluid fractioning device capable of selectively fractioning blood flowing out of the bone marrow to thereby retain a fraction of the blood; and (b) returning a non-retained fraction of the blood to circulation of the subject thereby filtering blood thereof.

According to still further features in the described exemplary embodiments the fraction of the blood includes water and solutes.

According to still further features in the described exemplary embodiments (a) is effected by implanting a port in a bone of the subject, the port being in fluid communication with the fluid fractioning device.

According to still further features in the described exemplary embodiments the fluid fractioning device is implanted in soft tissue of a body of the subject.

According to still further features in the described exemplary embodiments the fraction of the blood retained by the fluid fractioning device is routed to a bladder, a Genito-Urinary system or a reservoir via a fluid conduit positioned between the fluid fractioning device and the bladder, the Genito-Urinary system or the reservoir.

According to still further features in the described exemplary embodiments the reservoir is a bag disposed outside the body of the subject.

According to still further features in the described exemplary embodiments the fluid fractioning device and reservoir are placed outside the body of the subject

According to still further features in the described exemplary embodiments the bone marrow is iliac bone marrow.

According to still further features in the described exemplary embodiments the subject suffers from chronic kidney disease (CKD) and/or renal failure.

According to still further features in the described exemplary embodiments the subject suffers from congestive heart failure.

According to yet another aspect of the present invention there is provided a system for treating blood of a subject comprising: (a) a bone port configured for establishing fluid communication with a bone marrow of the bone: (b) a blood treatment device being in fluid communication with the bone port and being for modifying a property of blood flowing therethrough; and (c) a return port being in fluid communication with the blood treatment device and being for returning the blood to a circulation of the subject.

According to still further features in the described exemplary embodiments the blood treatment device includes cells or molecules capable of treatment of hepatic failure, synthesis of insulin, or synthesis of EPO or any other therapeutic protein.

According to still further features in the described exemplary embodiments the cells or molecules are encapsulated by a semi-permeable barrier.

According to still further features in the described exemplary embodiments the cells are selected from the group consisting of hepatocytes, endothelial cells, renal tubular cells, renal glomerular cells, pancreatic beta cells, neural cells, endothelial cells, fibroblasts.

According to still further features in the described exemplary embodiments (c) is effected by returning the blood to the bone marrow of the bone.

According to another aspect of some embodiments of the present invention, there is provided a method for affecting a pathophysiological condition in a body of a live subject, the method comprising: connecting an inlet port of a bone bypass shunt to a first bone portion adjacent a first bone marrow; location such that an inlet port lumen of the inlet port facilitates fluid communication with blood accumulated or flowing at the first blood marrow location; connecting an outlet port of the bone bypass shunt to a second bone portion adjacent a second bone marrow; location such that an outlet port lumen of the outlet port facilitates fluid communication with at least one of bone marrow, a formed cavity, and bone marrow vasculature, located at the second bone marrow location; via the inlet port lumen, removing a chosen volume of blood from the first bone marrow location; and via the outlet port lumen, delivering at least part of the chosen volume of blood to the second bone marrow location.

According to some embodiments of the invention, the method comprises choosing the first bone marrow location and the second bone marrow in a single bone. According to some embodiments of the invention, the method comprises choosing the first bone marrow location in a first bone, and choosing the second bone marrow location in a second bone other than the first bone.

According to some embodiments of the invention, the first bone and the second bone are of a same bone type. According to some embodiments of the invention, the first bone and the second bone are of different bone types.

According to some embodiments of the invention, choosing the first bone marrow location is based on an indication that red type bone marrow content is greater than yellow type bone marrow content.

According to some embodiments of the invention, the first bone marrow location is located at a flat bone or/and at an axial skeleton bone, or in one of a pelvis, a sternum, a cranium, a rib, a vertebra, and a scapula.

According to some embodiments of the invention, choosing the second bone marrow location is based on an indication that yellow type bone marrow is greater than red type bone marrow content.

According to some embodiments of the invention, the second bone marrow location is located at an epiphyseal end of a long bone, or vertebrae, or maxilla.

According to some embodiments of the invention, the method further comprises affecting or treating the chosen volume of blood before the delivering the chosen volume of blood. According to some embodiments of the invention, the affecting or treating includes at least one of: blood filtering, adding a medicament(s), adding or removing cells, adding or removing blood, and adding or removing a cytokine(s).

According to some embodiments of the invention, the pathophysiological condition is indicative of a systemic illness linked to failure of a bodily organ in the live subject. According to some embodiments of the invention, the systemic illness is selected from the group consisting of: chronic renal failure, chronic heart failure or fluid overload, osteoporosis, osteopenia, short stature, hyperparathyroidism, myelofibrosis an autoimmune disease, and a rheumatological disease.

According to some embodiments of the invention, removing and delivering are continuously maintained or sequentially repeated until there is indication of a local change in bone strength, thriving, or/and thickness, at a cortical bone portion adjacent the first bone marrow location or the second bone marrow location. According to some embodiments of the invention, removing and delivering are continuously maintained or sequentially repeated until there is indication of a systemic change in bone strength, thriving, or/and thickness. According to some embodiments of the invention, removing includes removing the chosen volume of blood into a proximal opening of a conduit of the bone bypass shunt, and delivering includes delivering the at least part of the chosen volume of blood from a distal opening of the conduit.

According to some embodiments of the invention, the first bone marrow location is at least 5 cm distant to the second bone marrow location.

According to some embodiments of the invention, the pathophysiological condition is indicative of a local illness in, or adjacent to, the first bone marrow location or/and in, or adjacent to, the second bone marrow location. According to some embodiments of the invention, the local illness is one of non-healing fracture, scoliosis, asymmetric facial bone growth, a bone lesion, osteoporosis, bone marrow dysplasia myelofibrosis, osteomyelitis, arid Pott's disease.

According to some embodiments of the invention, the method further comprises at least one procedure selected from the group consisting of: harvesting bone marrow, harvesting stem cells, and performing platelet pheresis.

According to some embodiments of the invention, the method further comprises attaining or/and maintaining chosen blood flow characteristics of at least one of: a chosen flow rate, a chosen total deliverable volume, and a chosen total duration of delivering blood. According to some embodiments of the invention, the chosen flow rate is within a range of 10 cc/min and 50 cc/min, or within a range of 50 cc/min and 100 cc/min, or within a range of 100 cc/min and 300 cc/min. According to some embodiments of the invention, the chosen total deliverable volume is within a range of 100 milliliters and 500 milliliters, or within a range of 0.5 liter and 1 liter, or within a range of 1 liter and 2 liters, or at least 2 liters. According to some embodiments of the invention, the chosen total duration is less than or equal to 3 hours. According to some embodiments of the invention, the chosen total duration is at least 3 hours.

According to some embodiments of the invention, the bone bypass shunt includes a compartment for holding a blood affecting means, the method further comprising placing the blood affecting means in the compartment so as to affect at least one of: blood pressure, blood temperature, blood chemical composition, and blood biological composition, of blood injected from the inlet port before the injected and affected blood reaches the outlet port.

According to some embodiments of the invention, the blood affecting means includes a blood filter configured for blood ultrafiltration. According to some embodiments of the invention, the blood affecting means includes a medicament.

According to some embodiments of the invention, the medicament is indicated for vascular tone management, and includes at least one of: an angiotensin-converting-enzyme inhibitor (ACE) inhibitor, an angiotensin receptor blocker (ARB), a vasodilator(s), a nitrate(s), and a calcium channel blocker. According to some embodiments of the invention, the medicament is indicated for heart rhythm control. According to some embodiments of the invention, the medicament is indicated for bone homeostasis, and includes at least one of vitamin D, calcium, parathyroid hormone, osteocalcin, TSH, growth hormone, estrogen, testosterone, Bone morphogenic protein(s), an immune modulator(s). Transforming Growth Factor (tgf)-beta or anti TGF-beta, and anti TNF-alpha blocker. According to some embodiments of the invention, the medicament is indicated for bone marrow modulation, such as erythropoietin, CSF, and GM-CSF.

According to some embodiments of the invention, the blood affecting means includes an added quantity of cells, that includes stem cells, blood, blood cells, cytokines, prostaglandins, nitric oxide, cyclic AMP, calcium, or/and TGF beta.

According to some embodiments of the invention, the bone bypass shunt includes a coupling means for coupling to a dialysis machine, the method further comprising connecting a dialysis machine to the coupling means and operating the dialysis machine for facilitating the removing of the blood or/and the delivering of the blood.

According to some embodiments of the invention, the method further comprises retaining a fraction of the chosen volume of blood or related components and routing the blood volume fraction to a bladder, a Genito-Urinary system, or a reservoir.

The present invention successfully addresses the shortcomings of the presently known configurations by providing a blood fractioning and treatment system capable of filtering or treating blood flowing through the bone marrow. The benefits of the bone marrow filtering include robust blood flow and pressure, a solid organ for the fixation of devices, and prevention of systemic emboli.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE SEVERAL VIEWS OF THE DRAWINGS

The invention is herein described, by way of example only, with reference to the accompanying drawings. With specific reference now to the drawings in detail, it is stressed that the particulars shown are by way of example and for purposes of illustrative discussion of the exemplary embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 schematically illustrates one embodiment of the present system, in accordance with some embodiments of the present invention;

FIG. 2 schematically illustrates another embodiment of the present system, in accordance with some embodiments of the present invention;

FIG. 3 schematically illustrates an exploded view of a blood fractioning device having a single filter assembly, in accordance with some embodiments of the present invention;

FIG. 4 schematically illustrates an exploded view of a blood fractioning device having a double filter assembly, in accordance with some embodiments of the present invention;

FIG. 5 is a schematic cross sectional view of one embodiment of the present system, in accordance with some embodiments of the present invention;

FIG. 6 schematically illustrates an exploded view of an electrically charged/polarized blood fractioning device constructed, in accordance with some embodiments of the present invention;

FIGS. 7A-7D schematically illustrate a prototype needle array port used in the pig study, in accordance with some embodiments of the present invention;

FIGS. 8A-8B schematically illustrate a prototype screw port used in the pig study, in accordance with some embodiments of the present invention;

FIGS. 9A-9B illustrate use of a jig for drilling holes for the needle array port of FIGS. 7A-D, in accordance with some embodiments of the present invention;

FIG. 10 illustrates the needle array port prototype attached in position over the iliac crest bone, in accordance with some embodiments of the present invention;

FIG. 11 illustrates blood flow out of the out port of the needle array port, in accordance with some embodiments of the present invention;

FIG. 12 illustrates blood flow out of the out port of the screw port, in accordance with some embodiments of the present invention;

FIG. 13 schematically illustrates the port-to-port mini circulatory system, of the present invention which was tested in a pig model, in accordance with some embodiments of the present invention;

FIGS. 14A-14B schematically illustrates a membrane filter assembly utilizable with the present invention, in accordance with some embodiments of the present invention;

FIG. 15 illustrates a prototype membrane filter assembly, in accordance with some embodiments of the present invention;

FIG. 16 is a graph illustrating the flow rate arid ultrafiltrate collection through a filter assembly including aNovasep membrane, in accordance with some embodiments of the present invention;

FIG. 17 is a schematic side cut view of an exemplary shunt, for delivering blood or for bypassing a blood flow from a first to a second bone marrow, in accordance with some embodiments of the invention;

FIG. 18 is a schematic side cut view of an inlet port or an outlet port of a shunt for delivering blood or for bypassing a blood flow from a first to a second bone marrow, in accordance with some embodiments of the invention;

FIGS. 19A-19B are schematic side cut views showing an exemplary shunt for delivering blood or for bypassing a blood flow from a first to a second bone marrow, wherein the first and the second bone marrows are located on different positions within one and same bone (FIG. 19A), or wherein the first and the second bone marrows are each located on a different bone (FIG. 19B), in accordance with some embodiments of the invention; and

FIG. 20 is a flow diagram illustrating an exemplary method of treating a local or a systemic illness by flowing or delivering blood from, a first bone marrow to a second bone or bone marrow, in accordance with some embodiments of the present invention.

### DESCRIPTION OF SPECIFIC EMBODIMENTS OF THE INVENTION

The present invention relates to apparatuses and methods for recirculating bone marrow blood and more specifically to use of bone bypass shunts for affecting (changing, treating or/and maintaining) a pathophysiological condition in a body of a live subject, either a systemic or/and a local condition, such as by filtering or/and treating blood, by delivering medication, or/and by controlling dynamic properties of the recirculated bone marrow blood.

Apparatuses (systems or devices) according to the present invention can be configured for filtering and/or treating blood flowing through a bone marrow. In some embodiments, the present invention can be used to compensate for, or correct, systemic conditions such as failed or failing kidney or cardiac functions, diabetes, hepatic disease and failure, neurological disease, hematological, metabolic and respiratory diseases as well as provide for the replenishment of missing biomolecules. In some embodiments, the present invention can be used to compensate for, or correct, local conditions such as illnesses in/of local blood vessels, of local neural tissue, of local muscle or other soft tissue, of local bone or other hard/calcified tissue, or/and of a local organ.

Some additional exemplary embodiments of the present invention are directed to a ‘bone bypass shunt’ for directing (e.g., delivering or recirculating) blood from a bone marrow to a bone, or to another bone marrow, in a body part of a live subject (patient). For such exemplary embodiments, the bone bypass shunt corresponds to a more generalized, and a more generally applicable, apparatus compared to the herein illustratively described system for fractioning blood of a subject. Some additional exemplary embodiments of the present invention are directed to a method of treating a local or a systemic illness by flowing or delivering blood from a first bone marrow to a second bone or bone marrow in a body part of a live subject (patient). Some additional exemplary embodiments of the present invention are directed to a method for affecting a pathophysiological condition in a body of a live subject.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Numerous attempts have been made to devise devices which are free of the limitations of standard hemodialysis and peritoneal dialysis approaches.

Although some of these attempts have produced devices that can be effectively used in dialysis (an external process), non-biological implants which are not limited by clotting, infection, soft tissue damage and necrosis, bleeding, and shearing remain a long felt and unmet need.

PCT publication WO/2010/052705 to the present inventor describes a blood filtering device which is configured for filtering blood flowing through a bone marrow. The device includes a device body which is configured for partial or full implantation within a bone marrow of a subject and a filter for filtering the blood flowing through the bone marrow. The device of WO/2010/052705 filters the blood at the source, i.e. at the site of bone implantation to recover a filtrate which is then communicated to the genitourinary system (e.g. bladder) or outside the body.

The bone marrow is an immuno-privileged site and thus can be utilized for the introduction of materials foreign to the host. Such an example is disclosed in U.S. Pat. No. 6,463,933, describing a method for delivering a biologically active substance including: cells, tissues, nucleic acids, vectors, proteins or pharmaceutical compositions to a mammal, by introducing the substance into the bone or bone marrow.

While reducing the present invention to practice, the present inventor experimented with various filtering configurations in efforts to enhance the rate of filtrate generation from bone marrow blood.

As is further described herein, the present inventor has devised a system in which blood is directed from a bone marrow source to a blood circulation sink (e.g. artery, vein, bone marrow) to create an artificial circulatory loop. The system further includes a blood fractioning device (e.g. filter) for recovering a filtrate from blood flowing through the circulatory loop and/or a treatment device for modifying blood flowing through the artificial circulatory loop.

Thus, according to one aspect of the present invention there is provided a system for fractioning blood of a subject such as a human. The present system can be used for blood filtration and thus can be used to supplement or replace renal functions in CHF and CKD/ESRD.

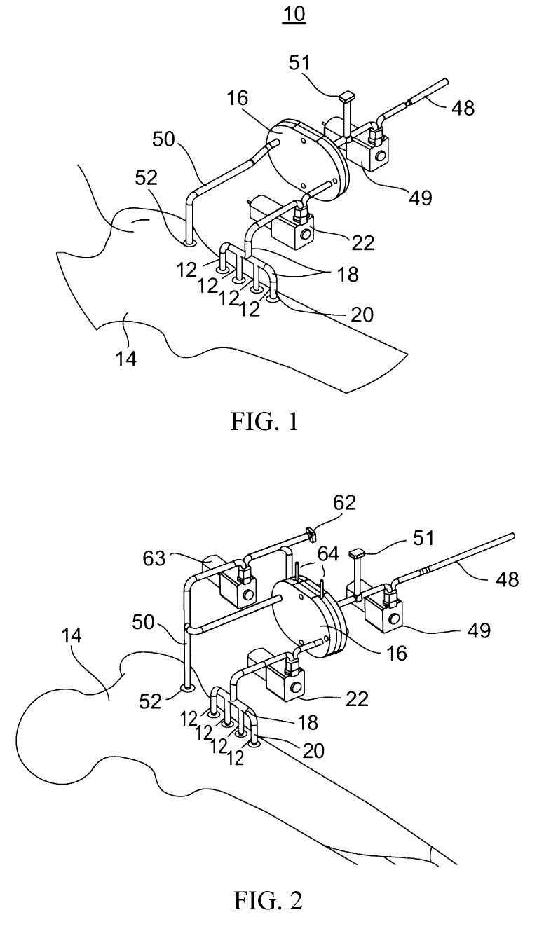

FIG. 1 illustrates one embodiment of the present system, which is referred to herein as system 10.

System 10 includes a bone (inlet) port 12 which is configured for establishing fluid communication with a bone marrow of a bone 14. Examples of bone 14 include, but are not limited to, the axial skeleton and those bones with hematopoietic function. Suitable bones include, but are not limited to, skull, vertebral bodies, iliac crest, rib, sternum long bones, hip, bones of the lower arm, and bones of the upper arm. Bones adjacent to or positioned above the patient bladder or GU system are preferred.

Any number of bone ports 12 can be used with the present system. Four bone ports 12 are shown in FIG. 1, however, a system 10 utilizing 1, 2, 3, 4, 5, 10, 50 or 100 bone ports 12 is also envisaged herein. Likewise, a system 10 including hundreds of needle-like bone ports 12 that traverse the cortex into the marrow are also envisaged herein. A system 10 configuration utilizing more than one bone port 12 can include an anchoring substrate (e.g. anchoring plate) for collectively anchoring a plurality of bone ports 12 connected to the substrate to bone 14.

Bone port 12 is configured as a hollow cylinder fabricated from biocompatible materials such as, for example, titanium, Nitinol, stainless steel, tantalum, SS 316L, Bio Dur 108 Alloy (Nickel free stainless alloy) or any other bone inert/integrative material. Bone port 12 can also be fabricated from a polymer such as Polypropylene, PTFE, ePTFE, PEEK, Nylon, poly ether-block co-polyamide polymers, polyurethanes such as aliphatic polyether polyurethanes, PVC, PAN, PS, polyethersulfone, polyethylene, polymethylmethacrylate (PMMA), polyhydroxylmethylmethacrylate (PHMMA), ceramics and the like.

Bone port 12 can be attached to a silicone or polymeric, (GoreTex™, PTFE, Polyurethane, Polyethylene, Polypropylene) conduit as is further described below.

Bone port 12 has a length, an outer diameter (OD) and an internal diameter (ID) suitable for establishing fluid communication between the bone marrow and an area outside the bone. Exemplary dimensions of bone port 12 can be OD of 1-10 mm, and ID of 0.2-8 mm. The bone port may have holes to enable blood and bone marrow to get into the port (e.g. hole diameter of 0.2-1.0 mm).

Bone port 12 can be attached to, or implanted within bone 14 via use of bone anchors, screw threads, staples, pins, glue and like. An array of bone ports 12 can also be used. Bone port 12 need not be fully implanted within the marrow region as long as fluid communication is established between marrow blood and a fluid opening at the top of bone port 12. Thus, partial implantation in which one end (distal) of bone port 12 resides within the marrow region and another end (proximal) resides outside the bone is also envisaged by the present inventor. Bone port 12 enables flow both from the bone and to the bone which allows for increased blood flow into the lumen of bone port 12. In that respect, the internal lumen of bone port 12 is configured with a surface which is smooth and highly polished (e.g. N4-average surface roughness 0.2 μm). The internal lumen may include structures for promoting cell attachment and growth (e.g. titanium beads or grooves/indentations within the internal surface). Such structures can be used to direct growth of cells and create compartments within the lumen which are at least partially surrounded by vascularized tissue.

The lumen can alternatively or additionally be coated with anti-fibrotic and/or anti-thrombotic substances such as rapamycin, sirolimus and the like, and/or with PEG polymers, surfactants, neutral polymers (e.g. poly(2-hydroxyethyl methacrylate), polyacrilimide, anionic polymers, phosphoryl choline polymers, gas discharge deposited coatings, self-assembly n-alkyl molecules with oligo-PEG head groups, self-assembly n-alkyl molecules with other polar head groups, natural Hydrophilic Surfaces (e.g. albumin, casein), polysaccharides (e.g. hyaluronic acid, heparin), liposaccharides, phospholipid bilayers or glycoproteins. Such coatings can also be used throughout bone port 12 and the remainder of the device when desirable.

Bone port(s) 12 is fluidly connected to a fluid fractioning device 16 via one or more fluid conduits 18. Fluid conduits 18 (as well as conduits 20, 48, 50 described below) can be fabricated from a polymer (e.g. silicone), a metal (e.g. stainless steel or titanium), GoreTex™ (PTFE) or combinations thereof. Conduits 18 are preferably elastic and designed to withstand an inner pressure of about 500 mmHg. In addition conduits 18 are designed to minimize or avoid kinking or alternatively include a bend-limiting element to avoid kinks. Conduits 18 preferably have an ID of 1-4 mm and an OD of 1.5-8.

In the configuration shown in FIG. 1, four bone ports 12 are connected to a single fluid conduit 18 via a manifold connected to four fluid outlets 20 (a four into one configuration).

In order to enhance blood flow from the bone marrow and into fluid conduit 18, system 10 preferably includes a device 22 for increasing a blood pressure gradient across bone port 12 (i.e. between the bone marrow and fluid conduit 18). Device 22 can be a pump 22 which creates a pressure differential of for example, 50-500 mmHg. Pump 22 is preferably a Peristaltic pump (e.g. Thomas SR10\30 or Welco wpm 1). A peristaltic pump is preferred since it provides several advantages: a) size and simplicity; b) the pump mechanism surrounds and squeezes the elastic tubing and enables pressure buildup with no additional mechanical parts within the tube, thus no direct contact of additional components or materials is made negating the risk of contamination; c) minimal components, no valves are required; d) small size, low energy; e) no energy required to maintain a holding position in either +/− pressure state; f) bi-directional flow ability; g) no flow disturbance; h) easy to sterilize and clean; i) can handle viscous and shear-sensitive fluids and prevent hackflow and syphoning without use of valves. Pump 22 can also be a pulsating pump such as Diaphragm pump (e.g. Schwarzer Precision model 270-EC-DB-L www.schwarzer.com/pages_en/produkt.phpid=95).

Pump 22 can also be a miniature impeller pump positioned within conduit 18. Such a pump can have a wide inlet and a narrow outlet configuration that can increase blood flow therethrough thereby enhancing blood flow from the bone marrow. Such a pump configuration can be fabricated from zirconium-niobium alloy and titanium-zirconium-niobium alloy as well as carbon coating techniques.

Blood flow from the bone marrow can also be enhanced by introducing ridges into the inner surface of conduits 18.

As is mentioned hereinabove, fluid conduit 18 routes blood from the bone marrow to a blood fractioning device 16.

Device 16 can be any device capable of fractioning blood flowing therethrough and retaining a fraction which is substantially devoid of cells and varying levels of proteins and ions depending on the anticipated usage of system 10 (e.g. serum with or without protein depending on filtering). Device 16 can fraction blood via centrifugation (vortex tube), polarization or filtration.

Device 16 can fractionate the blood using a single fractionation step or via several steps in which each step fractionates the filtrate obtained from the previous step.

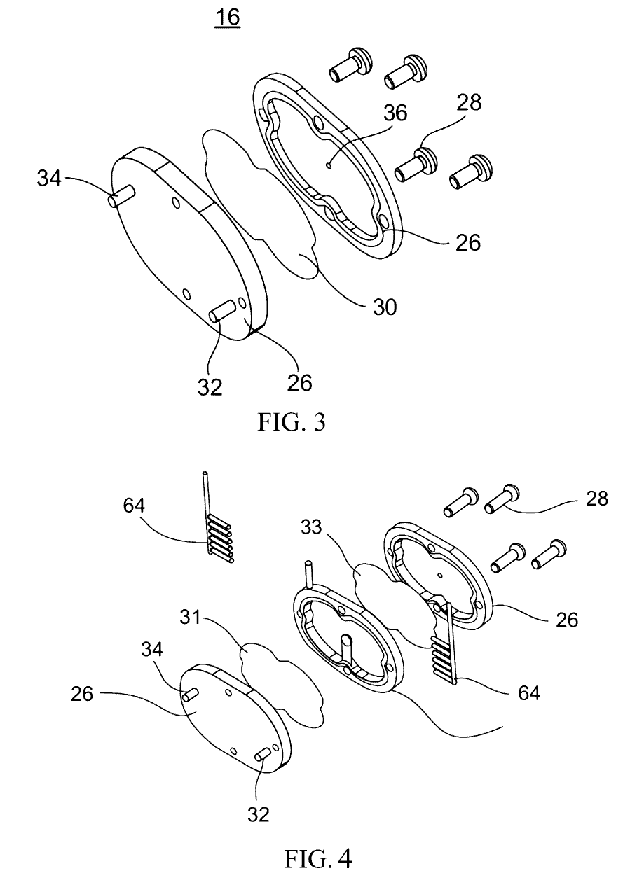

FIG. 3 illustrates one configuration of device 16. Device 16 includes a housing 26 (two halves fastened with screws 28, welded or glued) surrounding a filter 30. Housing 26 is fitted with input and output ports 32 and 34 (respectively) which conduct blood over filter 30 and a filtrate port 36 which conducts the fraction retained by device 16 to a target collection reservoir or vessel (further described hereinbelow). Housing 26 may be configured (internally) with channels/grooves that direct flow across the membrane (e.g. a housing with an internal spiral channel which directs fluid flow or any other configuration that can increase flow rate of blood past the filtration membrane). Another embodiment of device 16 is shown in FIGS. 14a-b and 15.

Filter 30 is permeable to water and solutes and impermeable to blood cells and biomolecules above a predetermined size (e.g. proteins, complex carbohydrates etc). Filter 30 preferably has a cutoff size selected from a range of 5-500 kDa. As such, filter 30 can restrict molecules above 5 kDa, 10 kDa, 15 kDa, 20 kDa, 25 kDa, 30 kDa, 35 kDa, 40 kDa, 45 kDa or 50 kDa from passing through the filter and being routed to the collection reservoir or vessel. Filter 30 can also include a combination of filters with different cutoffs. Filter 30 is preferably configured to withstand pressures of at least 400-500 mmHg or more (up to 1 ATM) without tearing and has a minimum surface area of 5 mm2, although larger surface areas in the range of 700 cm² are preferred. The surface area of filter 30 can be increased by rolling filter 30 into rods or by folding it into a three dimensional structure, or other techniques such as utilized in dialysis equipment, radiators, coolant systems or alike.

Filter 30 can be composed of any material suitable for such purposes, examples include metals, alloys, polymers, ceramics, biological material or combinations thereof.

Metal or alloy filters can be composed of stainless steel, nickel titanium alloys, cobalt-chrome alloys, molybdenum alloys, tungsten-rhenium alloys, liquid metal or any combination thereof.

Polymeric filters can be composed of poly acrylonitrile, polysulfone, polyethersulfone, polyethylene, polymethylmethacrylate, polytetrafluoroethylene (PTFE), polyester, polypropylene, polyether ether ketone, Nylon, silicone, polyether-block co-polyamide polymers, polyurethanes such as aliphatic polyether polyurethanes, polyvinyl chloride, thermoplastic, fluorinated ethylene propylene, cellulose, collagen, silicone or any combination thereof.

Filter 30 can be of a woven or non-woven configuration. Approaches for producing woven or non-woven filters are well known in the art.

Any number of filters can be used in device 16. FIG. 4 illustrates a two-filter configuration of device 16 in which the blood is filtered in a stepwise manner through a first filter 31 removing cells and then through a second filter 33 removing large biomolecules.

Two stage (also referred to herein as step-down) filtering is utilized to perform coarse filtration followed by fine filtration. Coarse filtration is effected using a 1 micron filter, fine filtration is effected using a 10-500 kD MWCO filter. This enables efficient energy usage since in the case of blood which includes solutes as well as cells it is easier to perform coarse filtration to remove most of the cells followed by a fine filtration of serum rather than whole blood.

Step-down filtering also enables collection of a serum like fluid (following coarse filtration) that can then be treated or ionically adjusted (e.g. solutes) in order to augment fluid reduction and adjust the patient's pH and ion concentration. Following such treatment, the coarse filtrate can be returned to the circulation as described herein.

Device 16 can be surgically implanted between the muscle and skin and connected to the ports as described above. Due to its proximity to the skin, device 16 can be periodically accessed (injection port) in order to inject various substances to clear filter 30 or to inject dialysate type solutions which may also enable dialysis potentially via sorbent dialysis systems such as Zeolite, conventional divalent exchanger, zirconium silicate or and other cation exchange system. The site of the filtration unit under the skin would also allow for inductive charging through the skin.

In order to reduce clogging (fouling) of filter 30, device 16 preferably includes a mechanism for clearing the surface of the filter or preventing fouling thereof. Such a mechanism can include a pump, a scraper/scrubber, an ultrasonic emitter and the like or a mechanism for creating an electrical or magnetic charge/polarization at or near filter 30.

FIG. 6 illustrates one configuration of a device 16 which includes a mechanism for creating an alternating electrical charge/polarization at filter 30. The Examples section which follows describes use of such a filter.

Device 16 includes a filter housing 38 which functions as a cathode, a filter 30, a sealing sleeve 40, an electrically insulated cover 42, electrically conductive rods 44 (preferably gold or alike plated) and assembly screws 46.

By polarizing the filtering surface of filter 30, charged or polarized blood constituents (e.g. glycoproteins) can be rejected or attracted in accordance with their electrical charge/polarization, thus enabling sorting of molecules and minimization of membrane fowling. AC polarization is more effective than DC polarization since proteins generally have a negative charge, while many solutes can have either a positive or negative charge. If only a negative charge is applied to the filter surface, the proteins will be repelled but cations such as Na++ or K++ etc will be attracted to the membrane and cause fouling. By using AC current, all the molecules are alternately attracted and repelled. The net effect of this attraction/repulsion of all molecules can keep the membrane clean and/or be used to clean the membrane.

Thus, system 10 routes blood from bone marrow through, or over, one or more filters (or alternative fractioning devices) to retain a filtrate that is largely composed of water and solutes. The water and solutes are then routed via fluid conduit 48 from a filtrate port 36 of device 16 to the Genito-Urinary (GU) system, bladder, or a reservoir disposed within or outside the body (e.g. collection bag). Conduit 48 can include a second pump 49 for increasing the pressure gradient between device 16 arid the target collection reservoir/tissue; pump 49 can be similar or identical to pump 22. The flow rate generated by pump 22 should preferably be much higher than the flow rate of pump 49 (The blood-filter contact time should remain low and flow velocity over the membrane surface should be high to diminish the risk of membrane fouling). Pump 49 can be used to generate a negative pressure while maintaining a relatively low flow rate through device 16. Pump 49 can also be used to generate backflow of fluid to clear filter 30. Conduit 48 can have a diameter ranging between about 1 to about 30 mm, preferably about 4 to about 10 mm and a length of about 50 to about 400 mm or preferably about 100 to about 200 mm.

The non-retained fraction (cells large biomolecules, water and solutes) can be routed back to circulation (preferably bone marrow or a blood vessel such as a vein artery).

The non-retained portion of the blood is routed back to circulation via fluid conduit 50 (diameter of 2-10 mm and a length of 15-100 mm) which is connected to a bone-anchored return (outlet) port(s) 52 or a blood vessel. When routed back to a bone (e.g. bone 14), return port 52 is preferably configured for fluid communication with a bone marrow of the bone. As such, return port can be of similar dimensions and materials as bone port 12. When routed back to a blood vessel, conduit 50 is connected to a return port which is configured as a standard synthetic graft to vessel anastomoses.

It will be appreciated that in the case of a system 10 which includes return ports 52 connected to bone marrow, the system can reverse flow by changing the pressure gradient, such that blood from the bone marrow will flow from, the bone marrow through port 12 to the filter and return to the bone through port(s) 52, or that blood from the bone marrow will flow through ports 52 and return to the bone marrow through ports 12). Such reversal of flow can be used to enable cleaning of the ports.

System 10 further includes pressure sensor 51 for monitoring the pressure at the receiving side of the membrane. The pressure data received from pressure sensor 51 enables system 10 to maintain the required pressure gradient by selectively operating pump 22 and/or 49.

FIG. 2 illustrates another configuration of system 10 which utilizes a two-stage filter, coarse filtering such as with a 1 micron pore size 0.2-4 micron followed by fine filtering 10-500 kD MWCO filter. Pump 22 creates constant blood flow across the first stage coarse filter 31. Pump 63 vacates the first stage filtered blood slowly back to the bone marrow while the pressure between the membranes is monitored by sensor 62. The filtrate of filter 31 flows across a fine filter 33 to generate a second filtrate which is evacuated from the body as described above. The primary filtrate can be then treated to preferentially and selectively remove additional solute such as potassium before allowing the primary filtrate to undergo further filtration which will then be directed to the GU system. While the proteinaceous primary filtrate will then shunt back into the circulatory system via the bone marrow or vessel. Such a two stage filtering approach is advantageous in that it may increase the life cycle of the filtering membranes and enable more thorough filtering. This configuration may also allow for increased treatment or modification of filtered serum such as selective and preferential excretion of solute, self generation of dialysate and acid/base adjustment.

The membranes may also contain protein secreting cells that are protected from the immune system and can be used to add proteins such as insulin, glucocerebrosidase, Factor VIII or other beneficial biomolecule. Renal tubular cells or hepatic cells that can be used for solute adjustment and detoxification of blood or serum can also be used with the present invention. Device 16 containing hollow fibers may be open to air in order to assist respiratory function. Pump 49 creates a pressure gradient across fine filter 33, this pressure gradient is monitored by sensor 51 and adjusted to enhance ultra filtrate flow-through filter 33 and into tube 48.

System 10 can further include a sensor or sensors (not shown) for sensing a concentrate of an analyte (e.g. glucose, BUN, Cr, Na, K, CI, HCO₃ pH etc) or for identifying blood parameters such as coagulation (e.g. blood viscosity and PT/PTT coagulation using a MEMs sensor such as that described in US20110302996) in the filtered bone marrow blood or in the recovered filtrate. The sensor(s) can be positioned, for example, within input port 32 or filtrate port 36 or along the conduits leading thereto. The sensor can utilize an amperometric enzyme probe, an optical probe or any other known probe suitable for measuring and generating an electrical sensor signal in response to analyte concentrations or to the parameters measured.

The system of present invention can also include a wireless communication unit (which can be positioned within bone port 12 or device 16) for communicating a filtration rate and optionally analyte sensor data to an extracorporeal control unit. The control unit can be used to control system operations such as filter defouling, pressure gradient (in the case of system 10 which includes pump 22 and/or 49) according to data communicated from sensors positioned in or on bone port 12, device 16 or conduits 18/48. Wireless communication and operation can be effected using RF, magnetic or ultrasonic communication approaches which are well known to the ordinary skilled artisan.

The present system can operate without any control over functions, or it can operate as a closed or an open loop system. In the closed loop configuration, the present system can incorporate a feedback loop which adjusts the pressure gradient according to the amount of water and solutes removed from the blood. The amounts of water removed can be measured via a sensor positioned within device 16 conduit 18/48 or the collection bag. Adjustment of the pressure gradient across filter 30 can then be controlled via a microprocessor positioned within device 16 and being in control of pump 22/49. In an open loop configuration, fluid flow sensor data can be sent to an extracorporeal processing and control unit. The processing unit can be first calibrated by a physician based on initial filtration rates. The processing unit can be recalibrated periodically (e.g. once or several times a year) if need be.

The present system may also include an indicator mechanism for alerting the subject or treating physician of filter 30 clogging or failure. Filter 30 clogging can be detected via an increase in pump 22/49 backpressure. Such an increase can be relayed wirelessly to an extracorporeal warning/control unit. Filter 30 failure can be detected by incorporating marker dyes into filter 30. Filter 30 breakdown would result in appearance of such a dye in the urine.

Although use of filter 30 clearing mechanisms such as those described hereinabove will ensure long duty cycle, use over extended periods of time (e.g. months, years) might necessitate filter replacement. In order to address such need, the present system preferable carries filter 30 or device 16 in a replaceable cartridge which can be exchanged via a minimally invasive procedure.

The present system provides several advantages when utilized in filtration of blood:
(i) minimizes contact of foreign body with bone marrow;
(ii) allows for increased surface area of filter with minimal drilling into the bone marrow, minimizing damage to the bone;
(iii) increasing flow past filter thereby increasing efficiency of filtration arid reducing membrane fouling;
(iv) step down filtration may set up the basis for more advanced solute and filtration adjustments, i.e., a fully functional kidney;
(v) mini-circulatory loop (from bone port to circulation) facilitates filter replacement;
(vi) the risk of clots being propagated systemically is significantly mitigated by re-introduction of retained fraction into bone marrow which would filter out any unwanted emboli prior to entering the general circulation.
(vii) flow past membranes or capsules may prevent fibrosis of membranes (hollow fiber or encapsulated) and be used for the immuno-protection of foreign cells;
(viii) fixation of device to bone more stable than blood vessel anastomosis; and
(ix) having an in/out port that can be reversed may ensure free flow of blood within the bone marrow and prevent fibrosis.

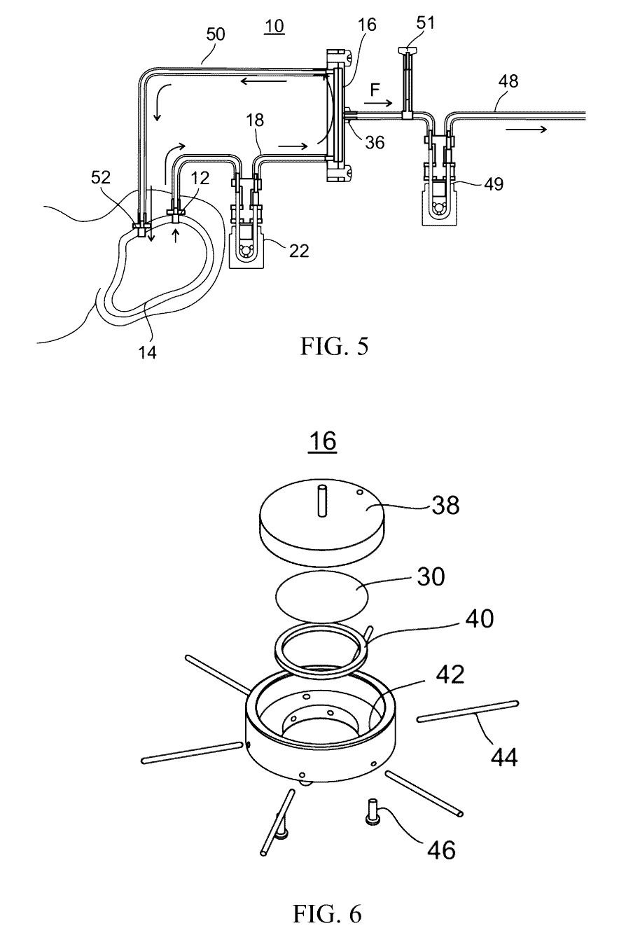

FIG. 5 illustrates the operation of system 10. Blood flowing out of bone marrow through bone port 12 (flow enhanced under the negative pressure applied by pump 22 is routed via conduit 18 into device 16. The blood is fractioned by one or more filter 30 into a filtrate (F) which flows out of port 36 of device 16 (under the negative pressure applied by pump 49). Conduit 48 carries the filtrate out of the body (into a collection bag) or into the GU system. The non-filtered portion of the blood is returned to circulation (e.g. bone marrow) via conduit 50 and return port 52.

As is mentioned hereinabove, system 10 of the present invention is highly suitable for use in treatment of fluid overload due to CKD/ESRD or CHF in the patient.

Given the fact that the flow of blood through the bone marrow can achieve a naturally occurring pressure gradient of 25-40 mmHg. The pump structure can increase the gradient to many hundreds of mm Hg or more (500 mmHg with pump 49) with respect to the collection reservoir or GU system, a minimum of 50 ml of water per 24 hr period can be removed by the present system. Such an amount of water would be sufficient for beneficial clinical effect especially for CHF patients.

Thus, the present invention also provides a method of treating CKD/ESRD or CHF. The method includes implanting within a patient in need the system disclosed herein.

A bone is exposed, and ports 12 and 52 inserted in to the requisite bone via drilling or tapping. The bone plugs or plate with ports are fixated to the bone and then the system components are attached. System components (filter 16, pump 22 and conduits 18) are connected to ports 12 and 52 and fixated to the bone or implanted in a subcutaneous pouch. The egress of filtered fluid is shunted to the GU system. The fluid may also be shunted to a collection device residing outside the body. Device 16 may be fixated to bone, implanted subcutaneously and may be designed as a cartridge that may be easily replaced. Device 16 may also be placed outside the body or provided with access to air.

The present invention can also be used to treat the blood flowing out of the bone marrow.

Thus according to another aspect of the present invention there is provided a system for treating blood of a subject. As used herein, the phrase “treating blood of a subject” refers to altering a property of blood on a physiological (e.g. solute concentration/balance), biochemical (removing or adding specific biomolecules) or cellular (removing or adding specific cell populations) level.

A system for treating blood includes a bone port (identical to bone port 12) which is in fluid communication with a blood treatment device (via a fluid conduit and optionally a pump such as those described for system 10). The blood treatment device is configured for modifying a property of blood flowing therethrough. The treated blood is then returned to circulation (e.g. bone marrow) via a return conduit and port(s) (such as those described for system 10). Thus, the blood treatment device of the present invention includes an artificial circulatory loop such as that described for system 10, but instead of filtering the blood flowing through such loop it treats it.

The blood treatment device can include cells, enzymes or other biologically active entities. The blood may be shunted though or around hollow fibers surrounded by, or containing cells. The cells can be renal tubular cells for treating kidney disease, hepatocytes for treating hepatic failure, beta cells for treating diabetes, nerve cells secreting dopamine for the treatment of Parkinson's or BDNF for the treatment of neurodegenerative disease, fibroblasts to secrete EPO or missing proteins such as Factor VIII or enzymes such as Glucocerebrosidase for Gaucher's disease.

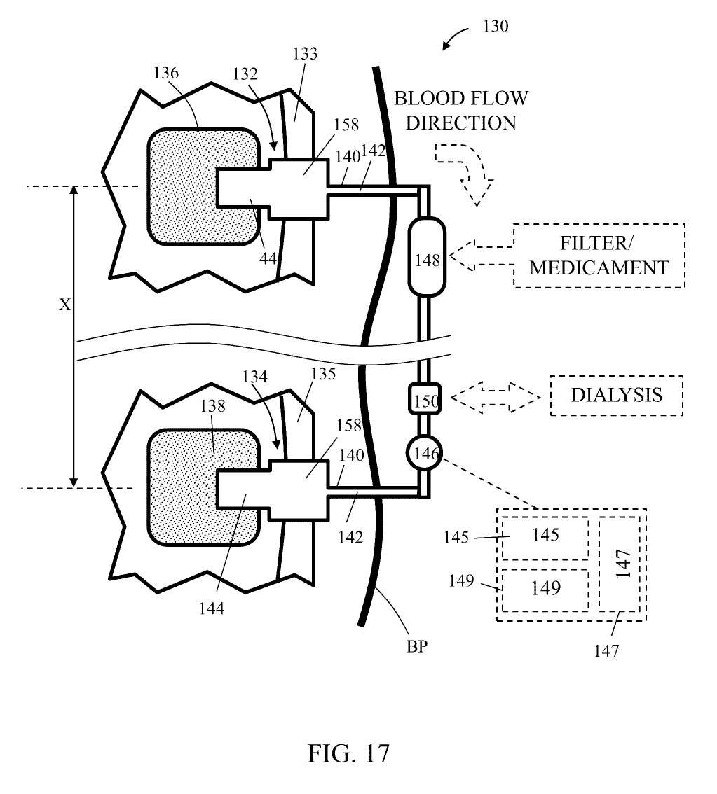

Hereinbelow are presented additional exemplary embodiments of the invention, with reference being made to FIGS. 17-20. Some additional exemplary embodiments are directed to a ‘bone bypass shunt’ for directing (e.g., delivering or recirculating) blood from a bone marrow to a bone, or to another bone marrow, in a body part of a live subject (patient). For such exemplary embodiments, the bone bypass shunt corresponds to a more generalized, and a more generally applicable, apparatus compared to the hereinabove illustratively described system for fractioning blood of a subject, for example, referenced as system 10 and shown in, or/and associated with, FIGS. 1-16. Some additional exemplary embodiments are directed to a method of treating a local or a systemic illness by flowing or delivering blood from a first bone marrow to a second bone or bone marrow in a body part of a live subject (patient). Some additional exemplary embodiments are directed to a method for affecting a pathophysiological condition in a body of a live subject.

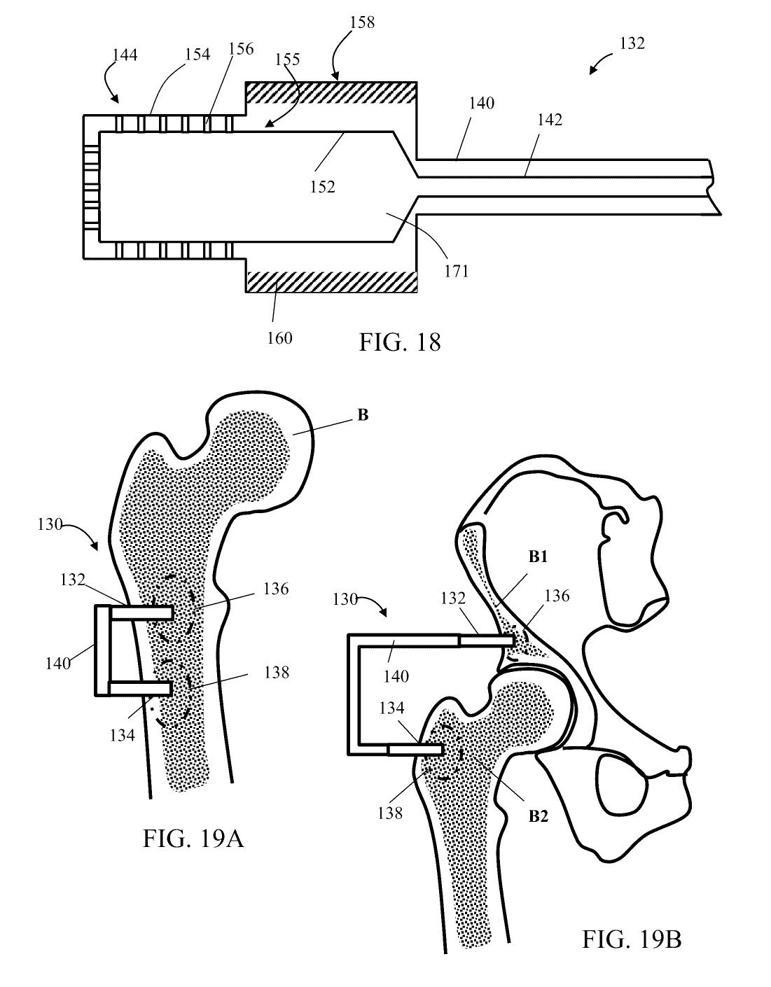

Referring to the drawings, FIG. 17 shows a schematic illustration of an exemplary-bone bypass shunt 130 for directing (e.g., delivering or recirculating) blood from a bone marrow to a bone or to another bone marrow, in a body part (BP) of a live patient (shown illustratively as bounded with an outline). Bone bypass shunt 130 may be structurally or/and functionally similar or identical at least partially to system 10 shown in previous figures. Shunt 130 includes an inlet port 132 enclosing a lumen 171 (shown in FIG. 18) and configured such that it may be connectable to a first cortical bone portion 133. Inlet port 132 may be positioned adjacent to or at first bone marrow location 136. Inlet port 132 is further configured to be in fluid communication with blood accumulated or flowing at a first bone marrow location 136.

Shunt 130 further includes an outlet port 134 enclosing a shunt lumen (optionally similar to lumen 171 of inlet port 132) and configured to be connectable to a second cortical bone portion 135. Inlet port 132 and outlet port 134 may be substantially similar in design, in shape or/and overall dimensions, or may be substantially different one with each other. Outlet port 134 is further configured to be in fluid communication with a second bone marrow location 138. Outlet port 134 may be positioned adjacent to or at second bone marrow location 138, such that an outlet port lumen thereof is in fluid communication with at least one of bone marrow, a formed cavity, and bone marrow vasculature. One of first bone marrow location 136 and second bone marrow location 138, or both, may include one of: a bone marrow, a formed cavity, and a bone marrow vasculature.

Shunt 130 further includes a conduit 140 having a passage 142 configured for allowing blood flowing from inlet port 132 to outlet port 134.

First bone marrow location 136 and second bone marrow location 138 may be present in a single bone. Optionally, alternatively or additionally, first bone marrow location 136 is present in a first bone, and the second bone marrow location 138 is present in a second bone, other than the first bone.

A distance X may be present between first bone marrow location 136 and second bone marrow location 138. Distance X may be about 5 cm or less. Alternatively, distance X may be more than about 5 cm. Inlet port 132 or/and outlet port 134 may be fully or partially embedded in bone/corresponding bones, whereas conduit 140 is mostly of fully provided outside bone(s). Optionally and as shown in FIG. 17, conduit 140 is partly or mostly expanding outside patient's body part BP, although, alternatively, conduit 140 may be partly, mostly or fully provided inside body part BP, for example extending in soft or/and fatty tissue. Further optionally, conduit 140 partly or mostly resides outside the patient's body and may include a chamber or cartridge for collecting blood. Optionally, conduit 140 is adjustable in length size to thereby fit to distance X present between first and second bone marrow locations 136 and 138, respectively.

Optionally, one or both inlet port 132 and outlet port 134 may include at least one chamber 144. Chamber 144 is configured for fluid communication with conduit 140 via a proximal opening of conduit 140.

Shunt 130 may further include a regulator 146 for regulating, and/or maintaining, and/or assessing, and/or alerting about, at least one blood characteristic. Optionally, the blood characteristics remain unchanged. The term “blood characteristics” refers to any physical, chemical or biological blood parameter or characteristic and include, but is not limited to pressure, flow rate, temperature, volume, cellular composition, and chemical composition. Regulator 146 is thus configured, inter alia, for regulating blood flow or blood composition characteristics. Optionally, alternatively or additionally, regulator 146 is configured for assessing and/or regulating the blood flow rate in the conduit 140 (in passage 142).

Regulator 146 may include or may be functionally linked with at least one of: a controller 145, an alarm 147, and a sensor 149. The sensor 149 may serve to assess at least one of a blood characteristics, selected from, but not limited to: blood pressure, blood temperature, blood flow rate, and a blood volume. The controller 145 may regulate each or at least one of those blood characteristics mentioned above. The alarm 147 may indicate a parameter-change or provide values of at least one of the above-mentioned blood characteristics.

In some embodiments, the shunt 130 is configured to test the blood to evaluate a blood parameter, for example, the glucose level, or coagulation profile (e.g., Prothrombin Time test (PT) and Partial Thromboplastin Time (PTT)).

In an exemplary embodiment, shunt 130 is further configured for maintaining a chosen blood flow characteristic(s). As used herein the term “a chosen blood flow characteristic(s)” includes, but is no limited to any blood characteristic that is chosen (for example by a physician or by a patient) to be regulated and maintained in advance. In an exemplary embodiment, the chosen blood flow characterisiic(s) refers to a chosen flow rate, a chosen total deliverable volume, a duration in which blood is delivered from first bone location 136 to second bone location 138, or a combination thereof.

In a further exemplary embodiment, the regulator 146 is configured to assess, regulate, and maintain the particularly chosen or predetermined blood flow rate within the shunt. The chosen flow rate may be, for example, within the range of: about 10 cc/min and about 50 cc/min, about 50 cc/min and about 100 cc/min, or between about 100 and about 300 cc/min. Each possibility represents a separate embodiment of the invention.

A total deliverable volume may also be chosen or predetermined. The chosen deliverable blood volume may be any value that equals to or in between the range of about 100 milliliters and about 40 liters. In an exemplary embodiment, the chosen deliverable blood volume may be, for example, within the range of about 100 milliliters and about 500 milliliters, about 0.5 liter and about 1 liter, or about 1 liter and about 2 liters. Each possibility represents a separate embodiment of the invention.

In a further exemplary embodiment, the chosen deliverable blood volume may be in the order of 50 liters, such as about 40 liters. Such deliverable blood volumes may be efficient, for example, for hemodialysis.

The total blood delivery duration may vary and depend on the disease or condition to be treated and may be, for example, a continuous treatment or a periodic treatment. The periodic treatment may be conducted for a duration of, for example, a few days, a few-hours, or a few minutes.

In some exemplary embodiments, shunt 130 is configured for treating a local illness, optionally adjacent or/and in same organ of first bone marrow location 136 or/and of second bone marrow location 138. The local illness may be any illness, disease, condition or syndrome that is amenable to changes in blood volumes in bones or/and elsewhere, in flow mechanics related to change in blood volumes in a bone marrow location, in blood characteristics and/or to bone marrow homeostasis, and/or a change that is considered to be related to a bone marrow. The local illness may be present in any bodily organ or tissue, including, but not limited to heart, kidney, bone, liver, brain, and bladder or bone itself

Particular local illnesses may include bone diseases such as, for example, a bone fracture (e.g., a non-healing), scoliosis, asymmetric facial bone growth, a bone lesion, osteomyelitis and Pott's disease.

Optionally, alternatively or additionally, shunt 130 is configured for treating a systemic illness. The systemic illness, disease, condition, or syndrome may be amenable to changes in blood volumes in bones or/and elsewhere, in flow mechanics related to change in blood volumes in a bone marrow location, in blood characteristics and/or to bone marrow homeostasis, and/or a change that is considered to be related to a bone marrow. Exemplary systemic diseases or conditions that may be treated by shunt 130 of the invention include, but are not limited to, chronic renal failure, chronic heart failure (CHF) or fluid overload, osteoporosis, osteopenia, short stature, hyperparathyroidism, and myelofibrosis.

In some embodiments, shunt 130 is further configured for maintaining chosen blood flow characteristics of blood flowing from bone marrow location 136 to bone marrow location 138 and thereafter this treatment to allow conditioning of either or both bone marrow location 136 and bone marrow location 138. The conditioning is selected from, but is not limited to a bone marrow harvesting, a stem cell harvesting, or a platelet pheresis. Those bone marrow conditionings may be utilized for donation or for treating essential thrombocytosis.

Shunt 130 may, but not necessarily, further include a compartment 148. Compartment 148 may be utilized, for example, for holding a blood affecting means such that blood flowing from inlet port 132 are subjected to particular treatment by the blood-affecting means before reaching outlet port 134. The blood affecting means may be means affecting a blood characteristic(s). In an exemplary embodiment, the blood affecting means may be means affecting blood pressure, blood temperature or a combination thereof. The blood affecting means may affect the chemical and/or the biological parameters of the blood.

In an exemplary embodiment, the blood affecting means include a blood filter configured for filtering blood (e.g., for ultrafiltration or hemodialysis related filtration). Blood filter may be a passive filter (e.g., blood flowing therethrough under naturally occurring pressure gradient), or an active filter (e.g., equipped with means such as pump for creating artificial chosen pressure gradient). The blood filter may be similar or identical structurally or/and functionally, at least partially, to previously described filter 30. The blood filter may include, included within or coupled with a blood fractioning device, which may be similar or identical structurally or/and functionally, at least partially, to previously described blood fractioning device 16. The blood filter may, for example, facilitate filtering particular cells type(s) or proteins. For example, the filter may have a cutoff size selected from a range of 5-500 kDa and as such may restrict molecules above a particular molecular weight. The blood fraction that passes through the filter continuous to flow to bone marrow location 138, whereas the non-passed blood fraction may either be routed back to the circulation or may be collected outside the body via an additional conduit connected to conduit 140 (which may be similar to fluid conduit 48, shown in FIG. 1, for example) and to the filter.

In a further exemplary embodiment, the blood affecting means include a medicament. As used herein, the medicament may be any drug or an agent indicated for treating the above mentioned local or systemic diseases, conditions, or syndromes. The medicament may be, for example, a vascular tone management or a hypertension medicament and may include, but is not limited to an angiotensin-converting-enzyme (ACE) inhibitor, an angiotensin receptor blocker (ARB), a vasodilator, a nitrate, a calcium channel blocker, or any combination thereof. The medicament may be a medicament for heart rhythm control. The medicament may be a bone homeostasis medicament, and may include at least one of: vitamin D, calcium, parathyroid hormone, osteocalcin, thyroid stimulating hormone (TSH), growth hormone, estrogen, and testosterone. The medicament may be for bone marrow modulation, such as erythropoietin, colony-stimulating factor (CSF) arid granulocyte-macrophage colony-stimulating factor (GM-CSF). The blood affecting means may further include an agent(s) such as a cytokine(s), a prostaglandin(s), a nitric oxide, a cyclic adenosine monophosphate (cAMP), calcium, transforming growth factor (TGF) beta, a Bone Morphogenic Protein (BMP), an immunomodulatory protein, or any combination thereof.

The blood affecting means may alternatively or additionally include cells (e.g., stem cells and blood cells) for adding to the blood flowing therefrom inlet port 132 to outlet port 134.

The shunt 130 may further include coupling means 150. Coupling means 150 may, for example, be utilized for coupling to a dialysis machine to thus allow dialyzing the blood flowing from the inlet port 132 to the outlet port 134. As used herein, a dialysis machine may be used for removing waste and excess water from the blood. The dialysis machine may be facilitated in cases of diseases or conditions of kidney failure.

Reference is now made to FIG. 18, which shows an exemplary variation of inlet port 132 (or/and of outlet port 134). Inlet port 132 and/or outlet port 134 may include chamber 144 enclosing at least one chamber cavity 155 with a chamber wall 154 configured with an inner surface 152. The inner surface 152 may have a surface finish of about 0.4 micrometer (equals to N5, in an N-Grade scale) or less, optionally 0.2 micrometer (equals to N4 in N Grade) or less. In some embodiments, the inner surface is configured to prevent cellular adhesion and minimize fibrosis, and it may be coated with in certain areas with bone growth inhibiting material. In some embodiments, the inner surface may be treated with sand blasting or 3D architecture to promote bone marrow ingrowth and attachment. In some embodiments, the inner surface may be coated with a drug eluting material that may contain compounds or agents capable of mediating a physiological effect(s). Chamber 144 is fluidly connected with conduit 140 having passage 142 that is configured for allowing blood flowing via inlet port 132 and outlet port 134.

Further optionally, chamber 144 is configured for fluid communication with its surroundings via at least one hole 156. The at least one hole 156 may have a diameter of 0.5 mm or less, 0.4 mm or less, 0.3 mm or less, 0.2 mm or less, or 0.1 mm or less. Each possibility represents a separate embodiment of the invention. Optionally, at least one hole may be greater than 0.5 mm in diameter, thus allowing bone marrow ingrowth.

Inlet port 132 and/or outlet port 134 may include a bone fixating portion 158 configured for fastening to and along a thickness of a cortical bone portion. The bone fixating portion 158 may be provided distally (as shown) or proximally to chamber 144. The bone fixating portion 158 may be any bone fixating means suitable for fixating the ports to the bone and may include, without limitation, raised helical rib(s) (thread(s)) 160.

Reference is now made to FIGS. 19A-19B which are schematic side cut views showing a simplified illustration of shunt 130 for directing blood flow from a first bone marrow location 136 to a second bone marrow location 138, according to some embodiments of the invention. In some embodiments, at least one of first and second bone locations 136 and 138, respectively is a bone marrow. In one embodiment, first bone location 136 is a bone marrow and second bone location 138 is selected from a bone marrow, a formed cavity, and a bone marrow vasculature.

Shunt 130 includes an inlet port 132 enclosing a lumen. Inlet port 132 is configured such that it may be connectable to a cortical bone portion (such as cortical bone portion 33 of FIG. 1). Inlet port 132 is further configured to be in fluid communication with blood accumulated or flowing at a first bone marrow location 136. Shunt 130 further includes an outlet port 134 enclosing a shunt lumen and configured to be connectable to a cortical bone portion (such as cortical bone portion 135 of FIG. 17). Outlet port 134 is further configured to be in fluid communication with a second bone marrow location 138.

Inlet port 132 may be positioned adjacent to or at first bone marrow location 136. Outlet port 134 may be positioned adjacent to or at second bone marrow location 138. Second bone marrow location 138 may be at least one of: a bone marrow, a formed cavity, and a bone marrow vasculature. Conduit 140 with passage 142 thereof is configured for allowing blood flowing from inlet port 132 to outlet port 134.

First bone marrow location 136 and second bone marrow location 138 may be present in a single and same bone B (FIG. 19A). Alternatively and optionally, first bone marrow location 136 is present in a first bone B1 and the second bone marrow location 138 is present in a second bone B2, other than the first bone (FIG. 19B).

Optionally, the first bone B1 and the second bone B2 have a same anatomical function. Alternatively, first bone B1 and second bone B2 may be bones having a different anatomical function.

Typically, a bone marrow consists of an adipose tissue which imparts a yellowish color (i.e., a yellow type bone marrow), and a hematopoietic tissue imparting a reddish color (i.e., a red type bone marrow). In various anatomical positions one can find certain ratios between the yellow and red type bone marrow. For example, in adults, bone marrows of the mid-diaphyseal portions of peripheral bones mostly consist of a yellow type bone marrow, whereas, in the general skeleton, the yellow and red type bone marrows are roughly equal in proportion. The axial skeleton retains hematopoietic potential throughout life. Children begin with hematopoiesis in all bones then the peripheral skeleton progressively converts to yellow marrow, returning to red marrow in times of certain diseases.

For example, first bone marrow location 136 may be chosen if it contains more red type bone marrow than yellow type bone marrow. Further, optionally, or additionally, second bone marrow location 138 may be chosen if it contains more yellow type bone marrow than red type bone marrow. Optionally, the first bone marrow location 136 and the second bone marrow location 138 may be chosen if they contain similar amounts of: yellow type bone marrow, red type bone marrow, or both.

In some embodiments, the amount or ratio of yellow and red type bone marrow may be assessed in advance. The step of assessing the amount or ratio of red and yellow bone marrow may include analysis of blood cells within the bone marrow, and/or the bone marrow structure (typically involves bone marrow aspiration and biopsy). In some embodiments, the locations of the first and second bone marrows are determined according to those assessments.

As detailed above, the first and second bone marrow locations 136 and 138 may be either on the same bone or on two different bones. In an exemplary embodiment, first bone marrow location 136 may be present on a fiat bone, or on the axial skeleton (bones with hematopoietic activity) optionally in one of: a pelvis, a sternum, a cranium, a rib, a vertebra and a scapula and second bone marrow location 138 may be located at or adjacent to an epiphyseal end of a long bone, optionally particularly within a femur bone, a humerus bone, a radius or an ulna.

Optionally, first bone marrow location 136 or/and second bone marrow location 138 may be present at or adjacent to a bone, selected from: a pelvis, a sternum, a cranium, a rib, a vertebra, a scapula, a femur, a humerus, a skull, a jaw, an epiphyseal end of a long bone (such as within a femur bone, a humerus bone, a radius or an ulna) and a mandibular condyle.

In some embodiments, first bone marrow location 136 is distanced from second bone marrow location 138 by about 5 cm or less. Alternatively, first bone marrow location 136 is distanced from second bone marrow location 138 by more than 5 cm.

The present invention further provides a method for treating a local or a systemic disease, the method including steps of removing a particular blood amount or flowing blood from a first bone marrow location (such as bone marrow location 136) arid delivering or flowing at least part of the blood to a second bone marrow location (such as bone marrow location 138).

As used herein the term “local illness” refers to any local illness, disease, condition or syndrome that is amenable to changes in blood characteristics and/or to bone marrow homeostasis, and/or that is considered to be related to a bone marrow. The local illness may be present in any bodily organ or tissue, including, but not limited to heart, kidney, bone, liver, brain, arid bladder.

In one embodiment, the local illness is a bone illness. Exemplary bone illnesses suitable within the context of the present invention, include, but are not limited to a bone fracture (e.g., anon-healing, and a non-union), scoliosis, asymmetric facial bone growth, osteomyelitis, Pott's disease and a bone lesion.

As used herein the term “systemic illness” refers to any systemic disease, condition, or syndrome that may be amenable to changes in blood characteristics and/or to bone marrow homeostasis, and/or that is considered to be related to a bone marrow. Exemplary systemic diseases or conditions that may be treated by the method of the invention include, but are not limited to, chronic renal failure, chronic heart failure (CHF) or fluid overload, osteoporosis, osteopenia, short stature, hyperparathyroidism, myelofibrosis an autoimmune disease, and a rheumatological disease.

The present invention further provides a method for treating a bone by promoting at least one of: encouraging bone strength, inducing bone growth, mediating bone thriving, the method including steps of removing or flowing a particular blood amount from a first bone marrow location (such as bone marrow location 136) and delivering or flowing the blood amount to a second bone marrow location, (such as bone marrow location 138). The method may include the steps of:
removing a chosen volume of blood from a first bone marrow location; and
delivering at least part of the chosen volume of blood to a second bone marrow location.

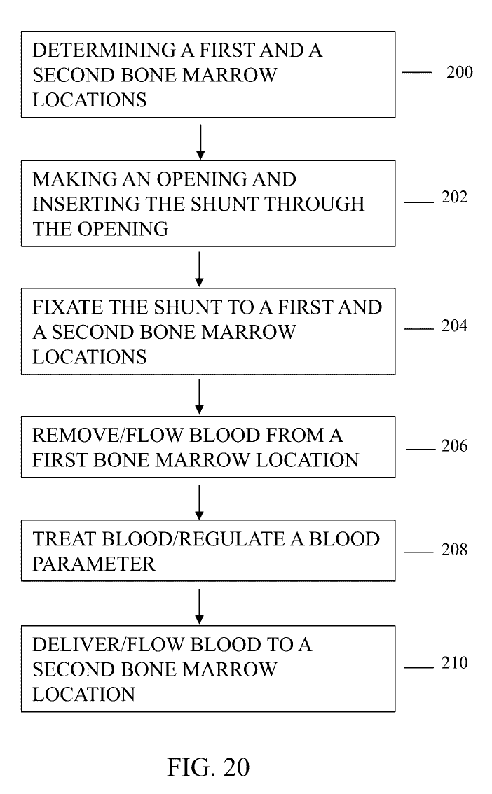

Reference is now made to FIG. 20 which illustrates a flow diagram of an exemplary method for delivering/flowing blood from a first to a second bone marrow location. The method may include a step 200 of determining, in advance, each of first bone marrow location (such as bone marrow location 136) and second bone marrow location (such as second bone marrow location 138). Typically, the determining the bone marrow locations is done by a physician and may be in accordance with the bone marrow parameters (such as amount or ratio of yellow vs. red type bone marrow) arid the disease or condition to be treated. The first and second bone marrow locations 136 and 138, respectively, may be present in a single and same bone B (see FIG. 19A). The first bone marrow location 136 may be present in a first bone B1 and the second bone marrow location 138 may be present in a second bone B2 (see FIG. 19B) being other than the first bone. In accordance with this embodiment, first and second bones B1 and B2, respectively, may be for example of the same bone type or may each be of a different bone type.

The method may further include a step of determining at least one of: red type bone marrow content and yellow type bone marrow content. The method may further include a step of choosing a first bone marrow location that is characterized in having a greater red type bone marrow content than yellow type bone marrow content. The method may further include a step of choosing a second bone marrow location that is characterized in having a greater yellow type bone marrow than red type bone marrow content. Optionally, at least one of the first and second bone marrow locations are determined following a biopsy taken from potential bone marrows and analysis thereof (e.g., the cellular composition and/or structure).

Optionally, first bone marrow location 136 may be present at a flat bone or at a skeleton, optionally particularly in one of a pelvis, a sternum, a cranium, a rib, a vertebra and a scapula. Further, optionally, or additionally, second bone marrow location 138 may be present at an epiphyseal end of a long bone, or vertebrae, or maxilla.

At step 202, the physician makes an opening (e.g., by an incision or a puncture) next to each of bone locations 136 and 138 and exposes the bone locations 136 and 138 via drilling or tapping and inserts shunt 130 through the opening. At step 204, the physician fixates the inlet and outlet ports, 132 and 134, respectively, through the bone fixation portion 158 to a cortical bone (possibly through threads 160). Total required fixation extent of the bone (inlet and outlet) ports may be achieved by allowing naturally occurring fibrosis or bone ingrowth onto portions of the bone ports.

At step 206, a chosen amount of blood is removed or a blood is facilitated to flow via inlet port 132 and from first bone marrow location 136.

In some embodiments, the step of removing and the step of delivering may each be sequentially repeated or continuously maintained until achieving the desired treatment or biological local or systemic effect. In some embodiments, the steps of removing and delivering are sequentially repeated or continuously maintained until obtaining an indication of a systemic effect or local change in bone strength, thriving or/and thickness.

The method may further comprise, but not necessarily, a step 208 of affecting or treating the blood, by blood affecting means, after removing, flowing or delivering blood from a first bone location 136 and before the blood flows or is being transferred to a second bone location 138.

The step of affecting or treating includes at least one of: filtering, adding a medicament, adding cells, adding blood, dialyzing and adding an agent (such as a cytokine).

Alternatively or additionally, step 208 may include regulating a blood by regulator 146 to thereby control, assess and/or alert a blood parameter or characteristic (such as blood pressure, blood temperature and blood flow rate.

At step 210, an amount of blood is delivered or blood flows to second bone marrow location 138.

Each of the following terms: ‘includes’, ‘including’, ‘has’, ‘having’, ‘comprises’, and ‘comprising’, and, their linguistic/grammatical variants, derivatives, or/and conjugates, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of’.

Each of the phrases ‘consisting of’ and ‘consists of’, as used herein, means ‘including and limited to’.

The phrase ‘consisting essentially of’, as used herein, means that the stated entity or item (system, system unit, system sub-unit, device, assembly, sub-assembly, mechanism, structure, component, element, or, peripheral equipment, utility, accessory, or material, method or process, step or procedure, sub-step or sub-procedure), which is an entirety or part of an exemplary embodiment of the disclosed invention, or/and which is used for implementing an exemplary embodiment of the disclosed invention, may include at least one additional ‘feature or characteristic’ being a system unit, system sub-unit, device, assembly, sub-assembly, mechanism, structure, component, or element, or, peripheral equipment, utility, accessory, or material, step or procedure, sub-step or sub-procedure), but only if each such additional ‘feature or characteristic’ does not materially alter the basic novel and inventive characteristics or special technical features, of the claimed entity or item.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as T, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably. For example, for stating or describing the numerical range of room temperature, the phrase ‘room temperature refers to a temperature in a range of between about 20° C. and about 25° C.’, and is considered equivalent to, and meaning the same as, the phrase ‘room temperature refers to a temperature in a range of from about 20° C. to about 25° C’.

The term ‘about’, as used herein, refers to ±10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub combination in the context or format of a single embodiment, may also be illustratively described and presented in the context or format of a plurality of separate embodiments.

Although the invention has been described in conjunction with specific embodiments thereof, it is evident that many alternatives, modifications, arid variations will be apparent to those skilled in the art. Accordingly, it is intended to embrace all such alternatives, modifications, and variations that fall within the spirit and broad scope of the appended claims.

All publications, patents, and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### EXAMPLES

Reference is now made to the following examples, which together with the above descriptions, illustrate the invention in anon limiting fashion.

### Example 1

### In Vitro Filtration of Blood Using an AC-Polarized Filter Assembly

The filter assembly described in FIG. 6 was used to filter fresh pig blood.

The testing system included a tank filled with 1 liter of fresh pig blood which is heated to 35-37 C oxygenated and stirred attached via a tube to the filter assembly. A vacuum pump capable of creating a vacuum of (−20)-(−70) mmHg was attached to a collecting tube attached to the filter assembly. Pressure is created within the tank for creating positive pressure of 30 mmHg inside the jar (resulting in a total of 50 mmHg pressure gradient across membrane). The filter cleansing was achieved via AC current applied at 200 Hz 1.5V with a duration of experiment being 8 hours. Filtration flow rate graph is obtained from the system's software and analyzed, the ultrafiltrate was sent for laboratory analysis. The results showed that as compared to straight filtration or as compared to blowback clearing, the AC current ultrafiltration membranes reduced the SD of filtration flow rate by half which indicates significant improvement in the efficiency of the filtration membrane which suggests that the membrane was kept cleaner.

### Example 2

### Pig Study

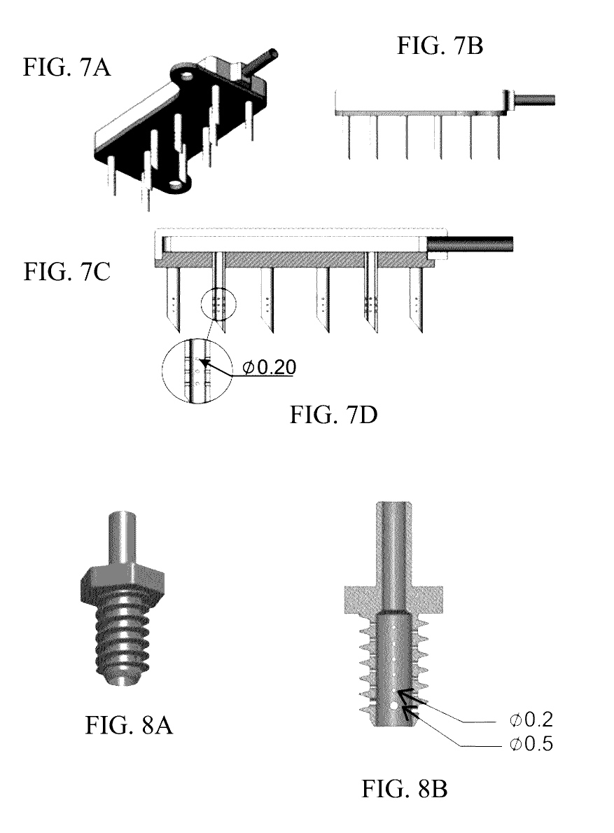

A pig model was utilized to test iliac crest bone marrow blood flow using several combinations of two port configurations, a needle array port (FIGS. 7a-d) and a screw port (FIGS. 8a-b). Several parameters were tested including dimensions, implantation process and sealing, as well as blood flow rate through the port with and without suction.

### Materials and Methods

Needle Array Port

A single 90 kg adult female pig was anesthetized and the iliac crest was exposed. Initially a 20,000 IU dose of Heparin was provided (i.v.) followed by a 12,500 IU dose 2 hours later.

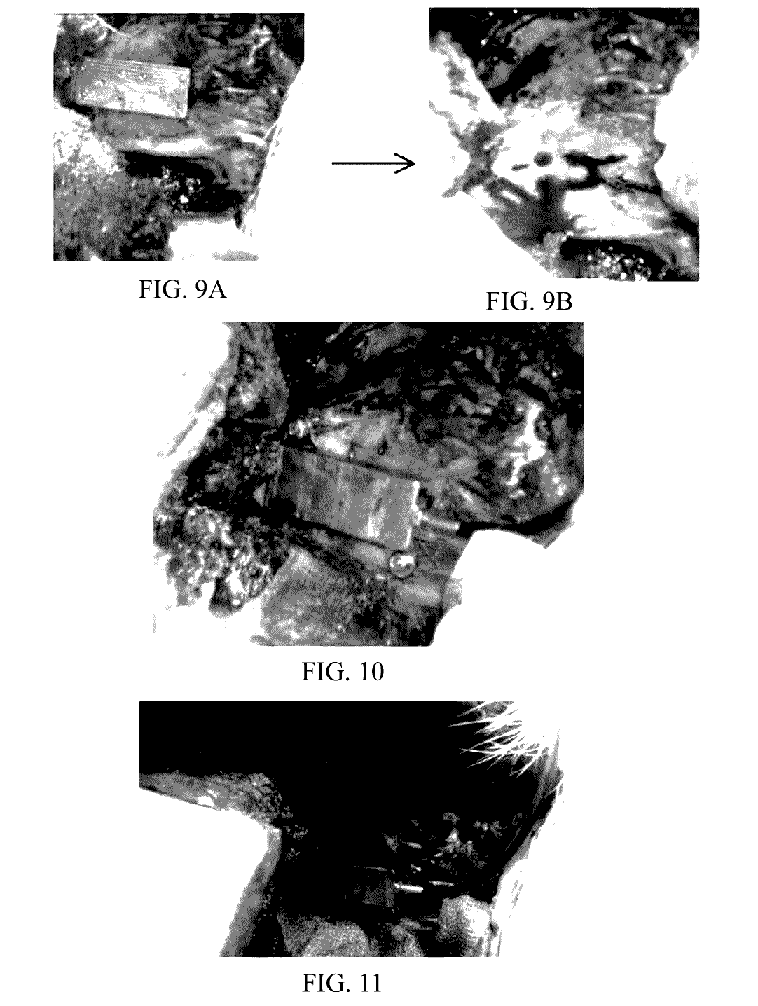

A jig (FIG. 9a) was used to create holes in the iliac crest (FIG. 9b) and a needle array including 10 needles, each having a length of 8.5 mm and 0.2 mm holes at the distal portion, was positioned with the needles inserted within the drilled holes (FIG. 10). No leakage of blood out of the needles was observed. The array plate was attached to the iliac crest via two screws. Blood flow through the out port of the array was observed following attachment thereof (FIG. 11). Without applying suction, blood flow through the out port tube was measured using a pressure sensor, measuring tape and a stopwatch.

Screw Port

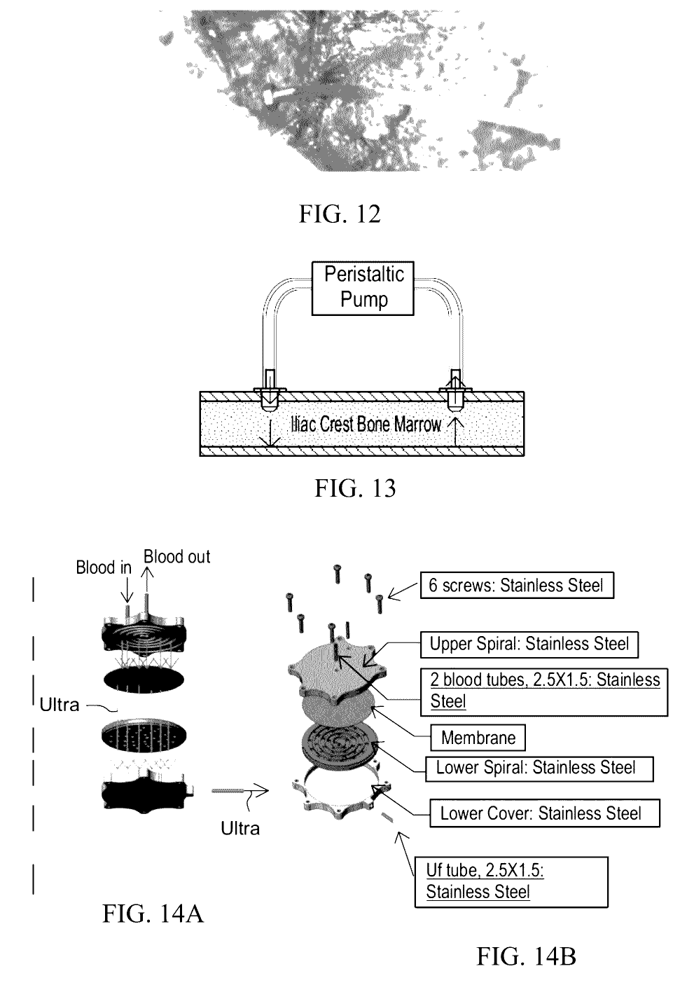

A hole was drilled in the iliac crest using a 3.5 mm drill bit. The screw port was implanted (FIG. 12) and no leakage of blood out of the port was observed. Without applying suction, blood flow through the out port tube was measured using a pressure sensor, measuring tape and a stopwatch.

An electronic pump unit (Thomas, 12V, Model 20300515), was connected to the out port of the needle array or screw port and suction was applied. Flow rate and pressure were measured by using a test tube and stopwatch or a dedicated software application which controls the pumps, receive readings (inputs) from the sensors (flow rate indication) and from the pressure sensors and store the experiment data.

Implantation of a Second Port

A second port (needle array or Screw type) was implanted as described about 5 cm away from the first implanted port (needle array or screw type), no leakage of blood out of the screw-port was observed. The two screw ports were fluidly connected through a peristaltic pump (FIG. 13) via their out ports in order to assess circulation.

Suction was applied resulting in blood flow from the Iliac crest bone marrow through one port toward the other port and back to the Iliac Crest bone marrow, flow-rate and pressure were measured.

Needle Array to Needle Array Circulation

Leakage around the needle holes was observed. Putty soft (Zhermack elite HD+) was used and appropriate sealing was achieved. Suction was applied resulting in blood flow from the Iliac crest bone marrow through one needle array towards the other Needle array and back into the Iliac Crest bone marrow, flow rate and pressure were measured.

Needle Array to Screw Port Circulation

Suction was applied resulting in blood flow from the Iliac crest bone marrow through one needle array towards the screw port and back into the iliac crest bone marrow, flow rate and pressure were measured.

Screw Port to Needle Array Circulation

Suction was applied resulting in blood flow from the Iliac crest bone marrow through the screw port towards the needle array and back into the iliac crest bone marrow, flow rate and pressure were measured.

In all cases, back flow was also measured.

### Results

The blood flow results with and without suction are summarized in table 1 below. The pressure measured for natural blood flow in bone marrow (without suction) is 30-32 mmHg.

TABLE 1

| | Flow rate | Flow rate |
|---|---|---|
| Blood flow through | [mm3/hour] | [ml/hour] |
| Needle structure | 18,095.5 | 18.0955 |
| without suction | | |
| (natural flow) | | |
| Screw-port without | 3,820.2 | 3.8202 |
| suction | | |
| (natural flow) | | |
| Needle structure with | 22,784.8 | 22.7848 |
| suction | | |
| Screw-port with | 17,734 | 17.734 |
| suction | | |
| Circulation: | | |
| Needle structure to | 19,200 | 19.2 |
| Needle structure | | |
| with suction | | |
| Circulation: | | |
| Needle structure to | 19,200 | 19.2 |
| screw-port | | |
| with suction | | |
| Circulation: | | |
| screw-port to Needle | 19,200 | 19.2 |
| structure | | |
| with suction | | |
| Circulation: | | |
| Needle structure to | 38,460 | 38.46 |
| Needle structure | | |
| with suction using 2 | | |
| pumps | | |

### Summary of Findings

Implantation of both screw and needle array -type ports was fast and easy. The blood flow rate through the needle structure was much higher than through the screw port. In both case, excellent bone marrow-to-bone marrow blood circulation was observed. A stronger pump (with a higher flow rate) can be used to increase the natural flow rate in cases where higher flow rates are desired. The pump system was effective at restoring full flow rates by blowing back obstructions that decreased flow.

### Example 3

### Filter Membrane Study

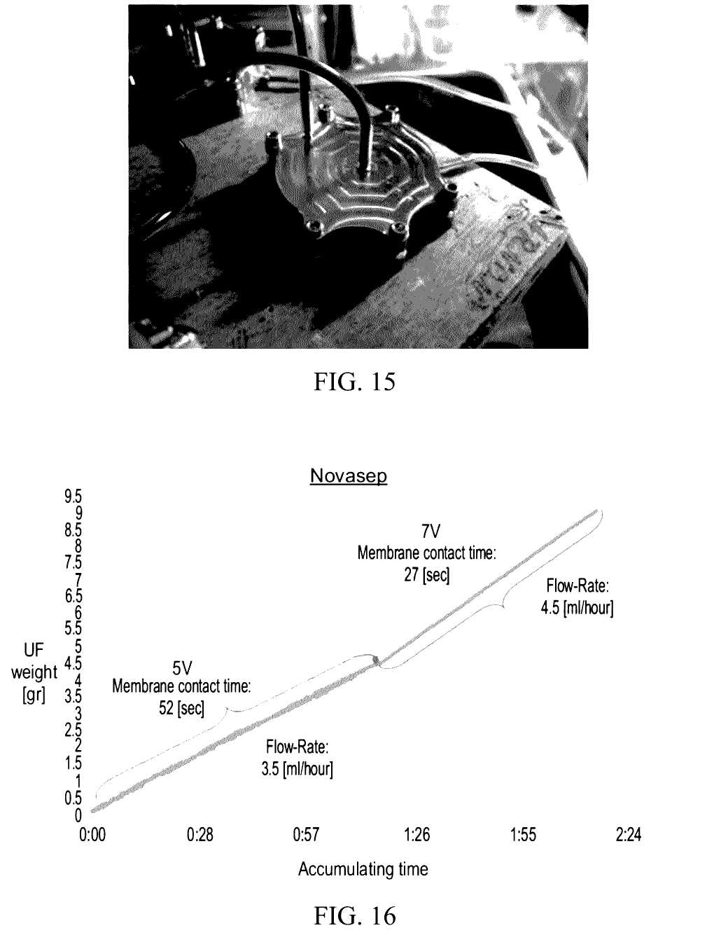

A filter assembly (FIG. 15) was constructed using the components illustrated in FIG. 14b. A Novasep Prostream 100 kDa membrane (effective filtration-1090 mm²) was clamped between two metal plates each having a spiral slot (with 364 mm total spiral length). As schematically shown in FIG. 14a, blood enters the upper metal plate through an inflow port and flows within the spiral slot over the membrane. The Ultra filtrate flows down towards a lower metal cover to be pumped out of the filter structure through a metal tube. The retained fraction is ejected out of the filter assembly from a metal tube positioned at the end of the spiral path.

Blood was pumped from ajar into the filter structure using a peristaltic pump (Thomas, 12V, Model 20300515). The blood flowed over the membrane within a spiral slot and back to the jar. Using a second peristaltic pump, the ultrafiltrate, which accumulated beneath the membrane, was pumped out from the filter structure into a sterile test tube. The test tube was placed on an electronic scale, which was connected to a computer and a graph representing ultrafiltrate as a function of time was generated.

FIG. 16 illustrates the graph obtained from Novasep Prostream membrane. The graph has two regions: input voltage of 5 [V] and input voltage of 7 [V]. As expected, the flow rate corresponding to an input voltage of 7 [V] is higher than the flow rate for an input voltage of 5 [V], At input voltage of 7 [V], an average of 4.5 [ml/hour] was obtained.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace ail such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method for affecting a pathophysiological condition in a body of a live subject, the method comprising:
connecting an inlet port of a bone bypass shunt to a first bone portion adjacent a first bone marrow location such that an inlet port lumen of said inlet port facilitates fluid communication with blood accumulated or flowing at said first blood marrow location;
connecting an outlet port of said bone bypass shunt to a second bone portion adjacent a second bone marrow location such that an outlet port lumen of said outlet port facilitates fluid communication with at least one of bone marrow, a formed cavity, and bone marrow vasculature, located at said second bone marrow location;
via said inlet port lumen, removing a chosen volume of blood from said first bone marrow location; and
via said outlet port lumen, delivering at least part of said chosen volume of blood to said second bone marrow location.

2. The method according to claim 1, comprising choosing said first bone marrow location and said second bone marrow in a single bone.

3. The method according to claim 1, comprising choosing said first bone marrow location in a first bone, and choosing said second bone marrow location in a second bone other than said first bone.

4. The method according to claim 3, wherein said first bone and said second bone are of a same bone type.

5. The method according to claim 3, wherein said first bone and said second bone are of different bone types.

6. The method according to claim 3, wherein said choosing said first bone marrow-location is based on an indication that red type bone marrow content is greater than yellow type bone marrow content.

7. The method according to claim 6, wherein said first bone marrow location is located at a flat bone or/and at an axial skeleton bone, or in one of a pelvis, a sternum, a cranium, a rib, a vertebra, and a scapula.

8. The method according to claim 3, wherein said choosing said second bone marrow location is based on an indication that yellow type bone marrow is greater than red type bone marrow content.

9. The method according to claim 8, wherein said second bone marrow location is located at an epiphyseal end of a long bone, or vertebrae, or maxilla.

10. The method according to claim 1, further comprising affecting or treating said chosen volume of blood before said delivering said chosen volume of blood.

11. The method according to claim 10, wherein said affecting or treating includes at least one of: blood filtering, adding a medicament(s), adding or removing cells, adding or removing blood, and adding or removing a cytokine(s).

12. The method according to claim 1, wherein the pathophysiological condition is indicative of a systemic illness linked to failure of a bodily organ in the live subject.

13. The method according to claim 12, wherein said systemic illness is selected from the group consisting of: chronic renal failure, chronic heart failure or fluid overload, osteoporosis, osteopenia, short stature, hyperparathyroidism, myelofibrosis an autoimmune disease, and a rheumatological disease.

14. The method according to claim 1, wherein said removing and said delivering are continuously maintained or sequentially repeated until there is indication of a local change in bone strength, thriving, or/and thickness, at a cortical bone portion adjacent said first bone marrow location or said second bone marrow location.

15. The method according to claim 1, wherein said removing and said delivering are continuously maintained or sequentially repeated until there is indication of a systemic change in bone strength, thriving, or/and thickness.

16. The method according to claim 1, wherein said removing includes removing said chosen volume of blood into a proximal opening of a conduit of said bone bypass shunt, and said delivering includes delivering said at least part of said chosen volume of blood from a distal opening of said conduit.

17. The method according to claim 1, wherein said first bone marrow location is at least 5 cm distant to said second bone marrow location.

18. The method according to claim 1, wherein the pathophysiological condition is indicative of a local illness in, or adjacent to, said first bone marrow location or/and in, or adjacent to, said second bone marrow location.

19. The method according to claim 18, wherein said local illness is one of non-healing fracture, scoliosis, asymmetric facial bone growth, a bone lesion, osteoporosis, bone marrow dysplasia myelofibrosis, osteomyelitis, and Pott's disease.

20. The method according to claim 1, further comprising at least one procedure selected from the group consisting of: harvesting bone marrow, harvesting stem cells, and performing platelet pheresis.

21. The method according to claim 1, further comprising attaining or/and maintaining chosen blood flow characteristics of at least one of: a chosen flow rate, a chosen total deliverable volume, arid a chosen total duration of delivering blood.

22. The method according to claim 21, wherein said chosen flow rate is within a range of 10 cc/min and 50 cc/min, or within a range of 50 cc/min arid 100 cc/min, or within a range of 100 cc/min and 300 cc/min.

23. The method according to claim 21, wherein said chosen total deliverable volume is within a range of 100 milliliters and 500 milliliters, or within a range of 0.5 liter and 1 liter, or within a range of 1 liter and 2 liters, or at least 2 liters.

24. The method according to claim 21, wherein said chosen total duration is less than or equal to 3 hours.

25. The method according to claim 21, wherein said chosen total duration is at least 3 hours.

26. The method according to claim 1, wherein said bone bypass shunt includes a compartment for holding a blood affecting means, the method further comprising placing said blood affecting means in said compartment so as to affect at least one of: blood pressure, blood temperature, blood chemical composition, and blood biological composition, of blood injected from said inlet port before said injected and affected blood reaches said outlet port.

27. The method according to claim 26, wherein said blood affecting means includes a blood filter configured for blood ultrafiltration.

28. The method according to claim 26, wherein said blood affecting means includes a medicament.

29. The method according to claim 28, wherein said medicament is indicated for vascular tone management, and includes at least one of: an angiotensin-converting-enzyme inhibitor (ACE) inhibitor, an angiotensin receptor blocker (ARB), a vasodilator(s), a nitrate(s), and a calcium channel blocker.

30. The method according to claim 28, wherein said medicament is indicated for heart rhythm control.

31. The method according to claim 28, wherein said medicament is indicated for bone homeostasis, and includes at least one of vitamin D, calcium, parathyroid hormone, osteocalcin, TSH, growth hormone, estrogen, testosterone, Bone morphogenic protein(s), an immune modulator(s), Transforming Growth Factor (tgf)-beta or anti TGF-beta, and anti TNF-alpha blocker.

32. The method according to claim 28, wherein said medicament is indicated for bone marrow modulation, such as erythropoietin, CSF, and GM-CSF.

33. The method according to claim 26, wherein said blood affecting means includes an added quantity of cells, that includes stem, cells, blood, blood cells, cytokines, prostaglandins, nitric oxide, cyclic AMP, calcium, or/and TGF beta.

34. The method according to claim 1, wherein said bone bypass shunt includes a coupling means for coupling to a dialysis machine, the method further comprising connecting a dialysis machine to said coupling means and operating said dialysis machine for facilitating said removing of said blood or/and said delivering of said blood.

35. The method according to claim 1, further comprising retaining a fraction of said chosen volume of blood or related components and routing said blood volume fraction to a bladder, a Genito-Urinary system, or a reservoir.
